(19) United States
(12) Patent Application Publication
(10) Pub. No.: US 2025/0161088 A1
(43) Pub. Date: May 22, 2025

(54) METHODS AND DEVICES FOR MEDICAL IMPLANTS

(71) Applicant: BIOMEDIX, S.A., Geneva (CH)

(72) Inventor: Norman GODIN, Geneva (CH)

(21) Appl. No.: 18/840,536

(22) PCT Filed: Jan. 25, 2023
(86) PCT No.: PCT/US2023/061211

§ 371 Date: Aug. 22, 2024

Related U.S. Application Data
(63) Continuation-in-part of application No. PCT/US2022/070759, filed on Feb. 22, 2022.

(51) Int. Cl. A61F 5/00; A61F 2/04

(52) U.S. Cl. CPC A61F 5/0036; A61F 5/0076; A61F 2002/044

(57) ABSTRACT

A stent configured to be placed in a patient to treat any of a variety of ailments can include a plurality of spaced-apart horizontal supports and a plurality of spaced-apart vertical supports. Each of the plurality of spaced-apart vertical supports can extend from a bottom-most one of the plurality of spaced-apart horizontal supports to top-most one of the plurality of spaced-apart horizontal supports. The stent can further include a plurality of spaced-apart connectors. Each connector can be positioned between a pair of the plurality of spaced-apart horizontal supports and a pair of the plurality of spaced-apart vertical supports.

### CROSS-REFERENCE TO RELATED APPLICATION

The present application is a continuation-in-part (CIP) of International Application No. PCT/US2022/070759, filed Feb. 22, 2022 and titled “METHODS AND DEVICES FOR MEDICAL IMPLANTS”, which claims priority to U.S. Provisional Application No. 63/200,212, filed Feb. 22, 2021 and titled “METHODS AND DEVICES FOR LONG-TERM ESO-GASTRO-INTENSTINAL IMPLANTS,” the disclosure of each is hereby incorporated by reference.

### FIELD

The presently disclosed technology relates generally to medical devices, prosthesis, and methods of using and/or implanting same. More particularly, one embodiment of the presently disclosed technology relates to methods and devices to place long-term implants in the wall and/or lumen of the esophagus of a patient to treat any of a variety of ailments, including but not limited to gastro-esophageal reflux disease and/or obesity, often without surgery.

### BACKGROUND

Obesity, for example, affects up to 40% of the world population. Removal of portions of the stomach and gastric by-pass are known treatments that are invasive and challenging procedures. Developing safe and relatively non-invasive methods of treatment could have an important impact on large segments of the population.

Further, reflux is an ailment that also affects a large portion of the world population. It is known to use a prosthesis to treat Gastro-Esophageal Reflux Disease (GERD) and/or to help a patient reduce their weight. Examples of such prior art devices are disclosed in WO 2019/155284, WO 2018/222819, and WO 2013/050381, which are hereby incorporated by reference.

Despite benefits, existing methods and devices used to treat reflux and/or obesity have drawbacks. For example, in WO 2019/155284, the mesh ring is not attached to the helical spring ring with sutures and the device tends to fall into the stomach before it can be integrated into the wall. In WO 2018/222819, food cannot pass between the wall of the esophagus and the device with a thicker ring since the mesh ring supporting the device is within the wall of the esophagus.

### BRIEF SUMMARY

While the prior art systems are beneficial in numerous ways, the systems, methods and devices of the presently disclosed technology provide benefits over what is currently known in the art.

In one embodiment, the presently disclosed technology provides a method of allowing integration of the mesh ring using platelet rich plasma (PRP) obtained from the patient's own blood and the mesh ring is integrated within the wall of the esophagus between the PRP added to the biopsy sites of the wall of the esophagus, which helps coagulation and “grips” the mesh on the external side of the mesh and esophageal cell wall stem cells that are obtained from the biopsies and are “recycled” and reinjected on the “internal” or luminal side of the mesh ring.

The most common reason for treatment of lesions of the esophagus is GERD. GERD is almost always treated with Proton Pump Inhibitors and/or antacids that block acid production so that the reflux is much less or not acidic anymore and the esophageal lesions heal. Prior to the presently disclosed technology, no one has bothered using PRP, as the cause of acid reflux, namely acid production, is not stopped and the esophageal lesions would recur very fast, the heartburn symptoms will not improve so there has been no good reason to use PRP in the esophagus until now. PRP and esophageal adult stem cells will help obtain a better integration of the mesh in the wall of the esophagus and the third and last ring (after the first ring compressing the wall of the esophagus or DM-1 (Diagnosis and Management 1) and the DM-2 (Therapeutic mesh ring), the DM-3 ring puts pressure on the area to help integrate the mesh supporting the tubular devices treating GERD and obesity in the wall of the esophagus.

All of this is done at standard endoscopy through the mouth, so no open or laparoscopic surgery.

In one optional embodiment, the presently disclosed technology is directed to a device and method used to implant a variety of Gastro-intestinal Anti-Reflux Devices (GARD™) placed minimally-invasively or non-invasively through the mouth of the patient to treat gastro-esophageal reflux disease and obesity with autologous biological compounds.

It is noted that the term GARD™ can refer to a Gastro-intestinal Anti-Reflux Device, as mentioned above, but the term GARD™ can also refer herein to a Gastroesophageal Anti-Reflux Device.

Optionally, the presently disclosed technology includes different versions of a technique to hold a stent within the esophageal wall, as the stent can help support new medical devices to treat first GERD and Obesity, as well as other applications in gastroenterology if the technique is applied in the duodenum or colon for example. Then a series of potential other applications in cardiology, pulmonary medicine, spine medicine, pancreatic surveillance and other applications can be considered with the development of AI and imaging techniques where different devices can be placed in the intermediary soft-wall and optionally an elastic wall silicon ring can be employed.

In one embodiment, the presently disclosed technology includes a stent configured to be placed in a patient to treat any of a variety of ailments. The stent can include a plurality of spaced-apart horizontal supports and a plurality of spaced-apart vertical supports. Each of the plurality of spaced-apart vertical supports can extend from a bottom-most one of the plurality of spaced-apart horizontal supports to top-most one of the plurality of spaced-apart horizontal supports. The stent can further include a plurality of spaced-apart connectors. Each connector can be positioned between a pair of the plurality of spaced-apart horizontal supports and a pair of the plurality of spaced-apart vertical supports.

Optionally, the device can be held in place for more than 6 months, and optionally from 3 to 5 years. For example, a stent can be configured to stay in place in the wall of the esophagus and help keep the Therapeutic-GARD™ (Th-GARD™) for this length of time safely in the esophageal lumen. Presently the DM1-GARD™ stays in place on its own up to 4 months in the esophageal lumen, but after that period of time falls into the stomach. A healthcare professional can determine if the stent can help hold the Th-GARD™ in place and the best method to do so. The therapeutic part (e.g., lamellar or tubular tube) can be removed and exchanged safely over time so when food or acid impacts the active part of the device, it can be exchanged.

### BRIEF DESCRIPTION OF THE DRAWINGS

The foregoing summary, as well as the following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments.

It should be understood, however, that the presently disclosed technology is not limited to the precise arrangements and instrumentalities shown. In the drawings:

FIG. 1 illustrates a Diagnosis and Management (DM) GARD™ according to one embodiment of the presently disclosed technology;

FIG. 2A illustrates a tubular-type GARD™ for GERD;

FIG. 2B illustrates a lamellar-type GARD™ for GERD;

FIG. 2C illustrates a reversed lamellar-type GARD™ for GERD during vomiting;

FIG. 3A illustrates one type of Obesity GARD™ according to one embodiment of the presently disclosed technology;

FIG. 3B illustrates another type of Obesity GARD™ according to one embodiment of the presently disclosed technology;

FIG. 3C illustrates yet another type of Obesity GARD™ according to one embodiment of the presently disclosed technology;

FIG. 4A illustrates normal layers of the esophageal wall of a human being;

FIG. 4B illustrates adult stem cells at the level of the basal membrane of the esophageal epithelium;

FIG. 5 shows a niche created by the ring of the DM-1 GARD™ compressing the different layers of the esophageal wall.

FIG. 6 illustrates different layers of the esophageal wall with the depth of penetration (level) of different methods of endoscopic resection of the esophageal wall;

FIG. 7 illustrates that after mucosal resection (X), Platelet Rich Plasma (PRP) with calcium gluconate are sprayed on the bottom of the bleeding niche. Resected mucosa (X) including stems cells is kept for later reinjection (not shown);

FIG. 8A illustrates one embodiment of the presently disclosed technology where a delivery catheter with a balloon that is used to deploy the mesh ring held on the delivery catheter with a stretchable magnetic bead ring;

FIG. 8B shows the inflated balloon pressing the mesh ring on the bleeding niche where PRP and calcium gluconate were sprayed to integrate the mesh ring in the coagulating blood;

FIG. 8C shows the magnetic bead ring that has been removed by pulling on threads and the net ring is pressed with the balloon against the wall of the niche. The slip knot on the tube is still in place and holds the GARD™ for GERD device allowing removal of the magnetic beads while keeping the position of the device;

FIG. 9A illustrates one embodiment of the presently disclosed technology, wherein instead of a balloon, a helical spring ring is placed inside the mesh ring and folded on the delivery, similarly to the introduction of the DM GARD™ Knots are holding the mesh ring on the helical spring to avoid any displacement of the mesh ring when the slip knots are pulled out and the helical spring is deployed;

FIG. 9B illustrates that the surgical threads holding the mesh ring will be cut or pulled endoscopically once the helical spring is deployed and the mesh ring is in contact with the patient's blood, PRP and calcium gluconate in the bottom of the niche;

FIG. 9C illustrates that the helical spring is delicately removed and pulled out leaving the mesh ring in place in contact with the coagulating mixture in the bottom of the niche;

FIG. 9D illustrates that the mesh ring is free in the bleeding niche with PRP and gluconate calcium. The slip knot holding the tubular part of the GARD™ for GERD is still in position to maintain device in place;

FIG. 10A illustrates that fragments of the biopsies of the epithelium cells obtained in the beginning of the procedure with or without cultured epithelial cells obtained when the DM GARD™ was placed originally are sprayed in a PRP/calcium gluconate solution on the “luminal” side of the mesh to reconstitute the esophageal epithelium;

FIG. 10B illustrates that the mesh ring is now “sandwiched” between the bottom of the coagulating niche and the reconstituted autologous epithelium of the esophageal wall FIG. 11A shows the different layers of the normal esophageal wall;

FIG. 11B illustrates the different depths of placement of the mesh ring depending how deep the mucosal resections are according to one embodiment of the present disclosed technology;

FIG. 12 illustrated a ring of one embodiment of the presently disclosed technology that is used at the end of the procedure to compress the area once the mesh has been covered with epithelial cells and the ring is left in position for a period of time before possible removal. All other remaining devices have been removed from the esophagus. The slip knot is removed from the tube and the tube opens;

FIG. 13A is a top perspective view of a stent or ring of one embodiment of the presently disclosed technology;

FIG. 13B is a side perspective view of the device shown in FIG. 13A;

FIG. 13C is a perspective view of the device shown in FIG. 13A attached or glued to a tubular part;

FIG. 14A is a top perspective view of a medical device or stent of another embodiment of the presently disclosed technology;

FIG. 14B is a perspective view of the device shown in FIG. 14A attached or glued to a lamellar or grooved tubular part;

FIG. 14C is a perspective view of the device shown in FIG. 14A attached or glued to a smooth tubular part;

FIG. 15A is a series of perspective views of a lamellar or grooved tubular part, the device shown in FIG. 14A, and a combination of the two;

FIG. 15B is a schematic perspective view of the device shown in FIG. 14A with points of suture or stitches to attach the device to a ring;

FIG. 15C is a schematic perspective view of an upper end of the device shown in FIG. 14A with surgical threads extending therethrough to attach to a ring according to one technique of the presently disclosed technology;

FIG. 15D is a schematic perspective view of an upper end of the device shown in FIG. 14A with surgical threads extending therethrough to attach to a ring according to another technique of the presently disclosed technology;

FIG. 16A is a perspective view of a lamellar or grooved tubular part having a magnetic wrap on a portion thereof;

FIG. 16B is a partially or completely magnetic medical device or stent in the style of that shown in FIG. 14A;

FIG. 16C is a side elevational view of a portion of a magnetic medical device or sent of one embodiment of the presently disclosed technology;

FIG. 16D is a cross-sectional view of a portion of several components of one embodiment of the presently disclosed technology in combination;

FIG. 16E is another cross-sectional view of a portion of several components of another embodiment of the presently disclosed technology in combination;

FIG. 17A is a cross-sectional elevation view of several components of one embodiment of the presently disclosed technology placed within a patient;

FIG. 17B is an enlarged, cross-sectional view of a portion of a device of one embodiment of the presently disclosed technology within the patient;

FIG. 18A is a perspective view of a magnetized ring being inserted into the device shown in FIG. 14A in accordance with one embodiment of the presently disclosed technology;

FIG. 18B is a perspective view of the ring inside the medical device shown in FIG. 18A;

FIG. 18C is a perspective view of a tube and helical ring being inserted into the combined device shown in FIG. 18B;

FIG. 18D is a cross-sectional view of a portion of the combination shown in FIG. 18C;

FIG. 19A is a perspective view of a non-magnetized ring being inserted into the device shown in FIG. 14A in accordance with one embodiment of the presently disclosed technology;

FIG. 19B is a perspective view of the ring inside the medical device shown in FIG. 19A;

FIG. 19C is a perspective view of a tube and helical ring being inserted into the combined device shown in FIG. 19B;

FIG. 19D is a cross-sectional view of a portion of the combination shown in FIG. 19C;

FIG. 20A is a perspective view of a lamellar or grooved tubular part having a magnetic wrap on a portion thereof and a helical spring attached thereto;

FIG. 20B is another version of a partially or completely magnetic medical device or stent in the style of that shown in FIG. 14A, wherein the magnets are located on the inside of the stent;

FIG. 20C is a side elevational view of a portion of a magnetic medical device or sent of one embodiment of the presently disclosed technology;

FIG. 20D is a perspective view of a Therapeutic GARD™ being inserted into the device shown in FIG. 20B; and

FIG. 20E is a perspective view of a Therapeutic GARD™ inside the device shown in FIG. 20B.

### DETAILED DESCRIPTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the words “may” and “can” are used in a permissive sense (i.e., meaning having the potential to or optionally) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

The method according to one embodiment of the presently disclosed technology includes GARDs™ placed through the mouth of a patient after calibration of the diameter of the patient's esophagus to select appropriately sized devices with a new Therapeutic Endoscopy technique to which autologous biological compounds are added, called a Therapeutic BIO-Endoscopy (TBE) procedure.

In one embodiment, first a DM GARD™ is placed to evaluate tolerance and efficacy but also to create a circular pressure niche in the esophageal wall of the patient. The DM GARD™ is then removed from the esophageal wall of the patient. Once the DM GARD™ is removed, the Therapeutic GARD™ device can be placed in the esophageal wall of the patient.

Therapeutic GARDs™ (e.g., the GARD™ for GERD and Obesity devices) are optionally made of 2 parts: a ring made out of a circular soft mesh in one embodiment or a stent, made out of metal or an alloy material like nitinol in another embodiment, and a tubular part. Collectively, the two parts comprise a structure sometimes referred to herein as a “prosthesis.”

In one embodiment, the stent is a woven, knitted, or braided mesh structure, optionally in the form of a cylinder. The stent can be made from any of a variety of materials, such as stainless steel, nitinol (nickel titanium), or chrome-cobalt alloy, for example. In one embodiment, the stent can be formed of any material that provides super elastic capacity for folding to pass the stent through the mouth of the patient and its elasticity when released to expand and reach the niche when the stent is released.

In one embodiment, the mesh ring is placed within the wall of the esophagus after localized resection of the esophageal wall using a series of biopsies or deeper resection with Endoscopic Mucosal Resection (EMR) or Endoscopic Submucosal Dissection (ESD). The resection causes bleeding and plasma or PRP prepared from the patient's blood is injected or sprayed (e.g., through a catheter) to speed up coagulation and healing. Gluconate calcium can optionally be added to the PRP so that the solution is more viscous and helps adhere better to the bleeding niche.

The mesh ring of the Therapeutic GARD™ is then pressed mechanically with either a balloon mounted on the introduction delivery system that presses the mesh ring on the coagulating mix of blood and PRP/calcium gluconate or a self-deploying helical spring ring on which the mesh ring supporting the tube is mounted.

In the balloon embodiment, the balloon is then slowly deflated and removed or the knots holding the mesh ring to the helical spring are cut or pulled out if slip knots have been used and the ring is delicately pulled back in the esophagus or out of the body through the mouth leaving the mesh ring in place.

The patient's epithelial stems cells and possibly fibroblasts are optionally removed with the biopsies taken during placement of the DM GARD™ and put in culture in a laboratory. Or, the stem cells can be obtained when the biopsies (or EMR/ESD) are taken from the niche at the beginning of the therapeutic procedure are then placed in a PRP/calcium gluconate solution and are sprayed on the mesh ring on the luminal side of the mesh that supports the tubular devices. This is done to help reconstitute the epithelial layers of cells removed previously at resection. A balloon on the delivery catheter is inflated and presses the epithelial stem cells in PRP on the mesh and/or a third ring is placed at the end of the procedure to exert pressure for a longer period of time than an inflated balloon place through the mouth can.

In one embodiment, the mesh ring is integrated in the wall of the esophagus between the esophageal wall side (external) where the bottom of the niche and the coagulating blood with PRP/calcium gluconate mix is located and the luminal side (internal) that is reconstituted with the patient's own epithelial stems cells from the biopsies added to the PRP/calcium gluconate solution. In this way, the surgical mesh is “sandwiched” in the reconstituted wall.

The surgical mesh can be made of several different non-resorbable compounds, such as but not limited to polypropylene, polyester, polyethylene terephthalate (PET), polytetrafluoroethylene (PTFE), polyvinylidene fluoride (PVDF) among others that are all used safely for many years in surgery as well as some mesh that include animal collagen, and mixtures thereof. A ring is added in the lumen of the esophagus at the end of the procedure to help put pressure and help heal the esophageal wall now holding the mesh ring supporting the Therapeutic GARD™ in the gastro-intestinal lumen.

This new approach to Therapeutic Endoscopy is called Therapeutic Bio-Endoscopy (TBE) as a biological component is added, namely the autologous cells of the esophageal wall that have been resected and then reinjected to reconstitute the esophageal wall after the mesh of the devices supporting all the devices has been placed in the esophageal wall in the patient's platelet rich plasma (PRP), which is known to help heal lesions in other areas of medicine or dentistry. When only fragments of the esophageal biopsies containing stem cells (that have not been previously placed in cell culture in the laboratory) are used with a PRP/calcium gluconate solution, a localized “in vivo” culture milieu to reconstitute the esophageal wall is created. Other tissues (e.g., bone, tendon, cartilage, skin, hair, etc. . . . ) have been regenerated using PRP and appropriate stem cells.

See (1) Yamada Y, Ueda M, Naiki T et al., Autogenous injectable bone for regeneration with mesenchymal stem cells and platelet-rich plasma: tissue-engineered bone regeneration. Tissue Eng. 2004 May-June; 10(5-6): 955-64. (2) Zhu M, Kong D, Tian R et al., Platelet sonicates activate hair follicle stem cells and mediate hair follicle regeneration, J Cell Mol Med. 2020 January; 24(2): 1786-1794. (3) Paoloni, J. et al., Platelet-rich plasma treatment for ligament and tendon injuries, Clin J Sport Med. 2011 January:21(1): 37-45. (4) Etulain J. Platelets in wound healing and regenerative medicine. Platelets 2018 September; 29(6): 556-568. The disclosure of each of these references is incorporated by reference. Table 1 below shows a summary of the GARD™ family of devices with the temporary DM-GARD™ placed first then removed and the Therapeutic-GARD™ separated in 2 families, the GARD™ device for GERD models and the Obesity GARD™ models for obesity.

TABLE 1
Family of GARD ™ of devices
6 models of GARDs ™ divided in 3 groups.

| | |
|---|---|
| I. | Diagnosis and Management (DM) GARD ™ |
| | (short term implant) |
| | Group 1. The DM-GARD ™ |
| | (1 model with several sizes of rings) |
| II. | Therapeutic GARDS ™ |
| | (long term implants) |
| | Group 2. GARD ™ for GERD. |
| | (2 models with several sizes of rings) |
| | The lamellar GARD ™ for GERD for mild to moderate GARD. |
| | The tubular GARD ™ for GERD for moderate to severe GERD. |
| | Group 3. The OBESITY GARDs ™ |
| | (3 models with several sizes of rings) |
| | OB1-GARD ™, obesity Class 1 (BMI 30 to 34.9) |
| | The GARD tubes reaches the stomach. |
| | OB2-GARD ™, obesity Class 2 (BMI 35 to 39.9) |
| | The GARD tube reaches the duodenum |
| | OB3-GARD ™, obesity Class 3 (BMI more than 40) |
| | The GARD tube reaches the jejunum |

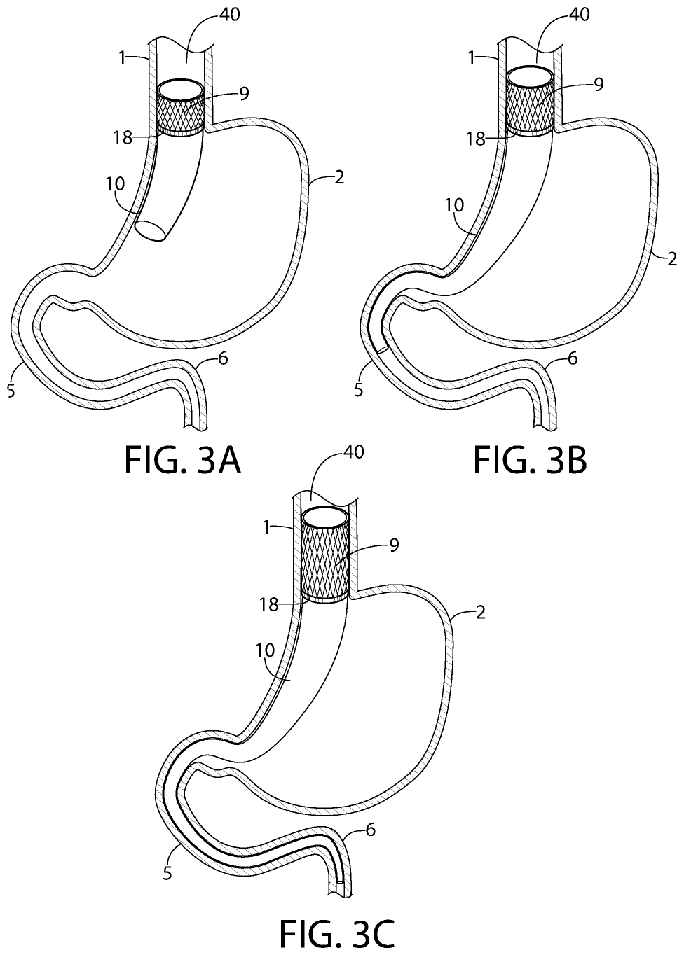

Various techniques are helpful to hold anti-reflux devices and anti-obesity devices in the lower esophagus of the living organism or patient. For example, the patient's adult stem cells can be “cultured” in vivo in the patient's own PRP obtained from the patient's blood. Another technique is to culture biopsies from the esophagus in an existing device made for cell culture/sorters in a lab. Using only components that can be reinjected in animals without immortalized cells, it is possible to culture their esophageal stem cells and demonstrate that in addition or instead of using in vivo cultures, one can use in vitro cultures in the lab taken from the animal, cultured and the cells that have multiplied can be used to recreate and/or repair an esophageal epithelium and thereby covering the nitinol stent holding the tubular devices for GERD (lamellar or tubular) and longer tubular devices as needed for the obesity GARDs™ (OB-1, OB-2 and OB-3), as shown in FIG. 3A-3C.

It is known to place staples at flexible endoscopy and perforate the wall of the esophagus to hold the GARD™ in place. One problem is that the staples can cause small holes through the different layers of the esophageal wall and secretions, often acid, passed from the lumen of the esophagus into the thorax and can cause mediastinitis in pigs.

Now, with the major development of laparoscopic surgery and even more with robotic surgery, it is possible to use laparoscopic technology to place a few sutures that have a greater range of motion and precision to place the sutures (2 to 10, preferably 3 to 5 sutures) using classical surgical curved needles through the abdomen than the classical laparoscopic techniques and give a better vision of the lower esophagus. When fluoroscopy is used additionally, it is easy for the healthcare professional to place his/her suture through the esophagus and through a nitinol stent, for example, to attach the stent securely to the wall of the esophagus. To avoid perforation as seen with metallic (e.g., nitinol) staples, the lumenal side as mentioned earlier can be covered with stem cells in PRP or provided by in vitro cultures as described earlier.

For the peritoneal/mediastinal sides of the sutures and the knot which the healthcare professional sees through the robot, a fibrin glue or fibrin sealant can seal the passage of the surgical thread through the wall of the esophagus as well as the knot tied by the healthcare professional on the “external” or peritoneal/mediastinal side) of the esophagus.

These techniques should prevent leakages and mediastinitis and/or peritonitis caused by placing sutures precisely under visual and X-ray control to suture the ring of the nitinol stent within the wall of the esophagus into the lumenal side. Benefits are also seen by placing the sutures from the laparoscopic side as well as using methods to “close” any “holes” caused by the passage of the surgical threads through the wall of the esophagus on the internal and external side, namely stem cells with PRP inside (lumen) and Tisseel (fibrin sealant) outside. A double approach can be employed, from inside the esophagus with endoscopy and outside the esophagus with laparoscopic surgery, preferably with a robot.

Another important method is to use the Apollo Endostitch device that allows a healthcare professional to do sutures through the mouth with a flexible endoscope and more recently with the Sx model using a classical one working channel endoscope, which most endoscopists use (instead of a 2 working channel endoscope that only very few endoscopists have as previously). It is now possible to secure the nitinol stent of the GARD™ in the wall of the esophagus using sutures placed through the mouth during placement of the Therapeutic GARD™ using the Apollo Endostitch Sx. This technique is beneficial as it can be done without surgery, through the mouth, on outpatients.

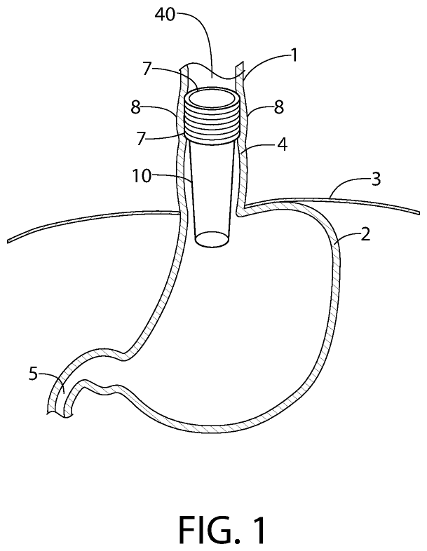

Referring now to the drawings in detail, wherein like numerals indicate like elements throughout, FIG. 1 illustrates the DM GARD™ placed in the esophagus (1) and the tubular (e.g., cylindrical and/or conical) part (10) reaching or extending into the stomach (2). The tubular part (10) is optionally flexible. Here, a hiatus hernia (4) often associated with moderate to severe GERD is shown. The diaphragm (3) separates the abdominal cavity underneath from the thorax above. The DM GARD™'s thick ring (7) holds in the esophagus mainly through pressure on the esophageal wall and makes the esophageal wall bulge creating a “niche” (8) that will be used to place the long-term Therapeutic GARD™ after the DM GARD™ has been removed. FIG. 1 also shows the lumen (40) of the esophagus.

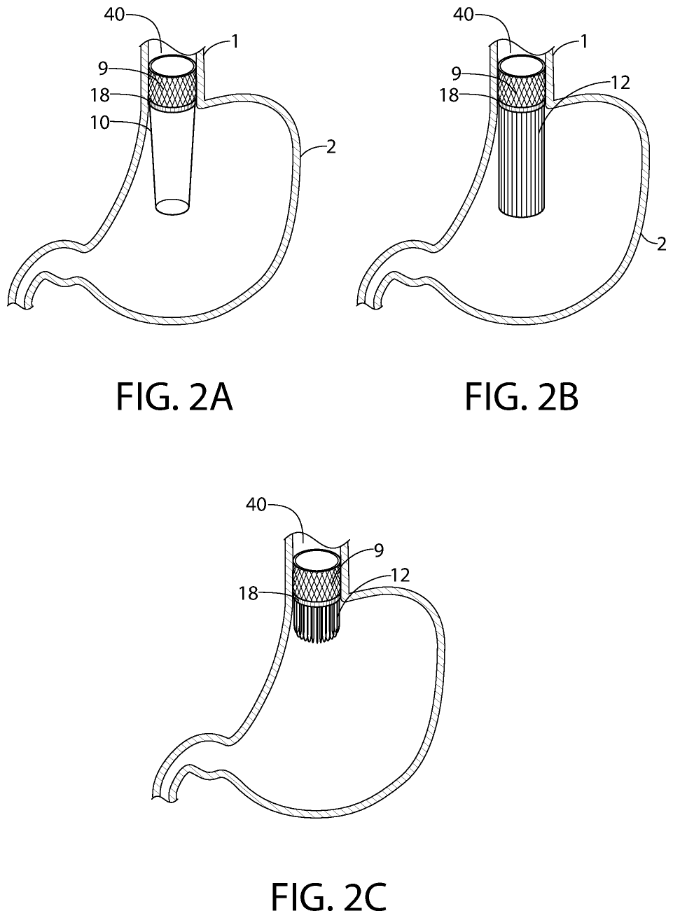

FIGS. 2A-2C illustrate the 2 main models used to treat GERD, namely in FIG. 2A, the GARD™ GERD device that blocks reflux and can also help patients who are overweight lose a few pounds. The GARD™ for GERD device blocks vomiting as surgery for reflux with its most common operation called the Nissen fundoplication also does. In FIG. 2B, the lamellar model (12) will also block reflux and allow vomiting when the lamellae under vomiting pressure turn back on themselves (FIG. 2C), but will have less effect on weight loss. An important feature is the radio-opaque zone (18) placed or located between the thinner mesh ring (9) and the tube (10) or the lamellae (12). This radio-opaque zone (18) will help locate the devices with fluoroscopy in case of need without having to do an endoscopy. FIGS. 2A-2C also show the lumen (40) of the esophagus. Optionally, the mesh ring (9) can be molded in silicone.

FIGS. 3A-3C illustrate 3 models of the Obesity GARD™. FIG. 3A shows the OB1-GARD™. FIG. 3B show the OB2-GARD™. FIG. 3C show the OB3-GARD™.

In FIG. 3A, the tube (10) of OB1-GARD™ device ends in, terminates in, and/or extends into the stomach and can be used for people having class 1 obesity, which is a Body Mass Index (BMI) of 30 to 34.9. In this device, the effect is mainly restriction to help lose weight, which means that people will have to eat smaller quantities and chew their food longer.

In FIG. 3B, the tube (10) of the device reaches the duodenum (5) so the device will cross or pass the whole stomach entering the stomach (2) where the lower esophagus (1) meets the stomach (2) and ending in the duodenum (5). This device mimics the effect of the “sleeve gastrectomy” where the healthcare professional cuts % of the stomach on the greater curvature side leaving a narrow band along the lesser curvature now occupied by the tube (10). Of course, with the OB2-GARD™, the whole stomach is spared and % of the stomach are not removed. The OB2-GARD™ can be used for patients who have class 2 obesity with a BMI of 35 to 39.9. Note that the mesh ring supporting the tube will have to be implanted over a higher surface of the esophageal mucosa and certainly deeper than for the GARD™ for GERD devices or the OB1 devices.

For morbid obesity or patients having BMIs of over 40, the OB3-GARD™ (FIG. 3C) can be placed with the tube ending in the jejunum (6). As in the surgical gastric by-pass, the OB3-GARD™ will help lose weight both by restriction as for the OB2-GARD™ but also by creating malabsorption as food will stay in the tube and will not be in contact with enzymes from the pancreas, or duodenum nor with bile before reaching the duodenum, in many ways similarly to the effect of gastric by-pass but again without surgery and surgical risk as the mortality of gastric by-pass is estimated to be about 1% because these patients are obviously high risk patients because of their morbid obesity. Again, the mesh ring supporting the OB3-GARD™ tube with have to cover a larger surface in the lower third of the esophagus and placed deeper in the esophageal wall. FIGS. 3A-3C also show the lumen (40) of the esophagus.

Table 2 below describes one method to obtain PRP from the patient's venous blood by centrifuging the blood twice. Other commercial centrifuges exist that allow for a single centrifugation, but usually the platelet concentration is lower than in this method and is therefore not recommended unless proven equivalent in platelet concentration.

TABLE 2
Platelet Rich Plasma Preparation.

| | |
|---|---|
| 1. | Obtain whole blood by venipuncture in acid citrate dextrose |
| | (ACD) tubes. |
| 2. | Do not chill the blood at any time before or during platelet |
| | separation. |
| 3. | Centrifuge the blood using a ‘soft’ spin. |
| 4. | Transfer the supernatant plasma containing platelets into |
| | another sterile tube (without anticoagulant). |
| 5. | Centrifuge tube at a higher speed (a hard spin) to obtain a |
| | platelet concentrate. |
| 6. | The lower 1/3^{rd} is platelet rich plasma (PRP) and upper |
| | 2/3^{rd} is platelet-poor plasma (PPP). At the bottom of the |
| | tube, platelet pellets are formed. |
| 7. | Remove PPP and suspend the platelet pellets in a small |
| | quantity of plasma (e.g., 2-4 mL) by gently shaking the tube. |
| 8. | Add 3% to 5% gluconate calcium to make the preparation |
| | viscous. |

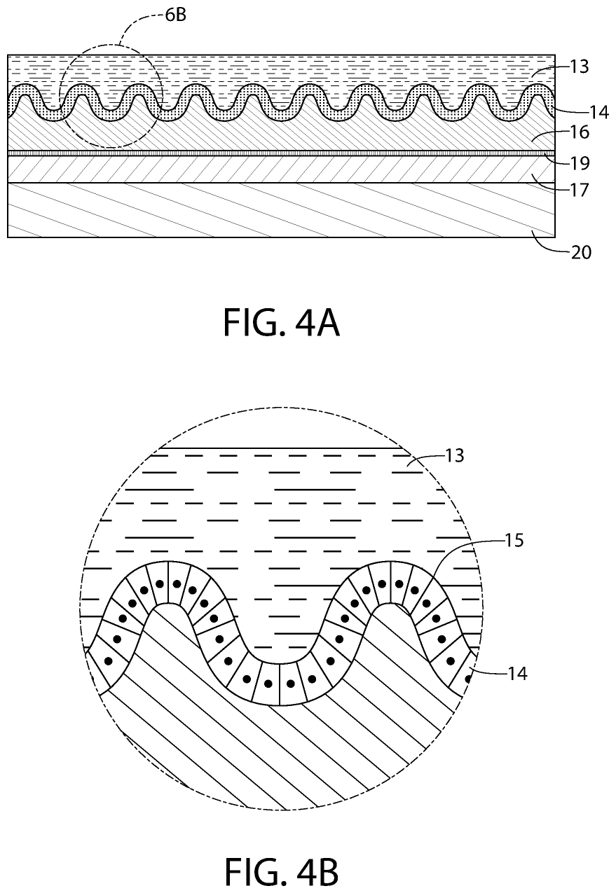

FIG. 4A illustrates schematically the different layers of the normal human esophageal wall. If one considers that the esophagus is a tube, the innermost layer in contact with food ingested through the mouth and passing through the esophagus into the stomach is the esophageal epithelium (13) and the outer most layer is a muscular layer that will “push” the food down into the stomach from the mouth using a progressive wave of contraction called the peristaltic wave (20).

The esophageal epithelium (13) is at the top of FIG. 4A. The bottom of the esophageal epithelium is similar to a wave called the basal membrane (14) that carries cells called adult stem cells (see element 15 of FIG. 4B) that play an essential role in repairing the esophageal epithelium if the wall is injured by disease or in our case by esophageal resection as in biopsies of the esophagus or deeper resections such as endoscopic mucosal resection (EMR) or endoscopic submucosal resection (ESD). The lamina propria immediately under the basal membrane (16) is part of the mucosa and the muscularis mucosae (19) separates the mucosa from the submucosa (17). The muscularis propria (20) itself has 2 layers with complex nerve systems (not shown). This demonstrates that the esophagus that appears to be a simple tube is in fact a much more complex organ than expected at first sight.

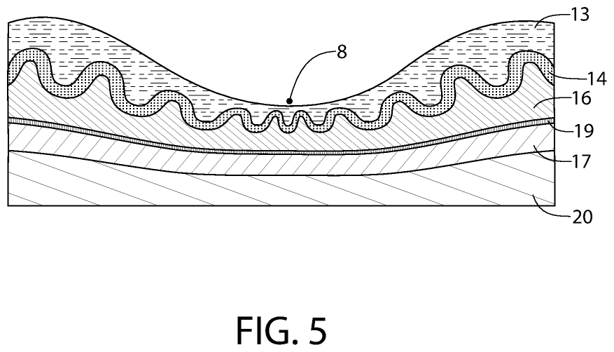

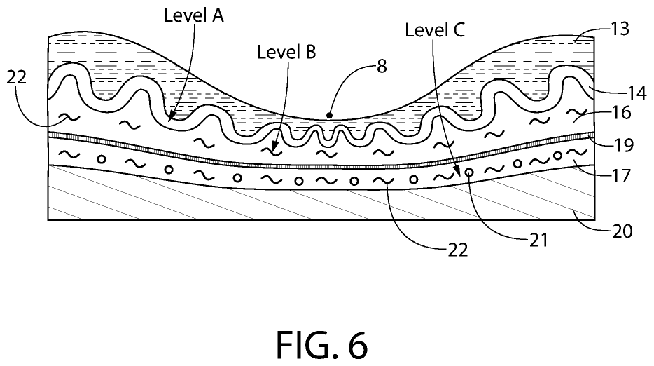

FIG. 5 illustrates the compression niche (8) left after removal of the DM-GARD™ and its thick ring that presses on the esophageal wall as shown in FIG. 1. This niche will facilitate the positioning of the much softer mesh ring of the Therapeutic GARD™. Elements 13, 14, 16, 17, 19, and 20 of FIG. 5 show the different layers of the esophageal wall that have been compressed by the ring of the DM GARD™ FIG. 6 illustrates the different depths of esophageal resection that can be used.

In most cases, Level A reaching the basal membrane with standard biopsies or level B reaching the lamina propria with endoscopic mucosal resection (EMR) should be sufficient to place the mesh of the Therapeutic GARD™ in position. Level C (submucosa) using Endoscopic submucosal dissection should only be used if longer, heavier tubes such as the OB2-GARD and OB3-GARD are needed to treat obesity since complications of using ESD are not unusual. Fibroblasts (22) from the lamina propria and the submucosa can also be cultured with the epithelial cells and reinjected once the mesh ring has been positioned in place. Blood vessels in the submucosa (21) are also shown. In certain cases, Level A and Level B can be combined by doing EMR and biopsies as well as Level B and Level C by doing EMR and ESD and some standard biopsies can be added.

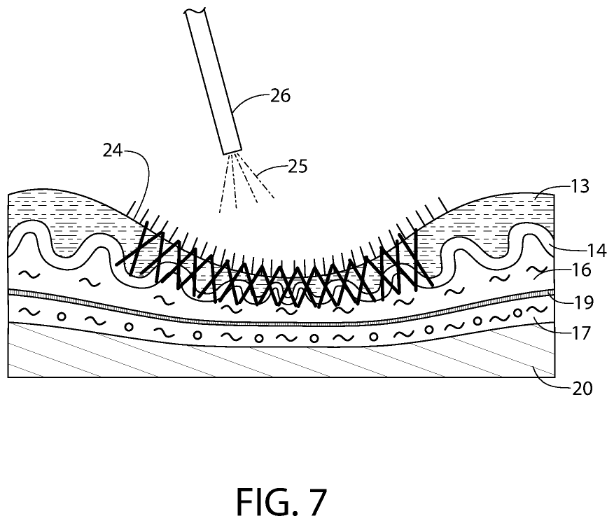

FIG. 7 symbolizes the partial resection of the wall of the esophagus with biopsies and/or EMR (24) and the bleeding (PRP) prepared at the beginning of the procedure with Calcium gluconate to make the PRP more viscous is sprayed (25) with a spray catheter (26) passed through the gastroscope (not shown).

Alternatively, to simplify the technique and reduce costs, 10-20 standard biopsies can be completed inside the niche inside the whole perimeter of the niche and after the stent is placed in the niche. Natural coagulation will bind the metal stent to the bottom of the niche with no need to use PRP, gluconate or blue methylene nor a fibrin glue such as Tisseel from Baxter. However, to secure the stent in the wall of the esophagus, these “biological agents” remain an option.

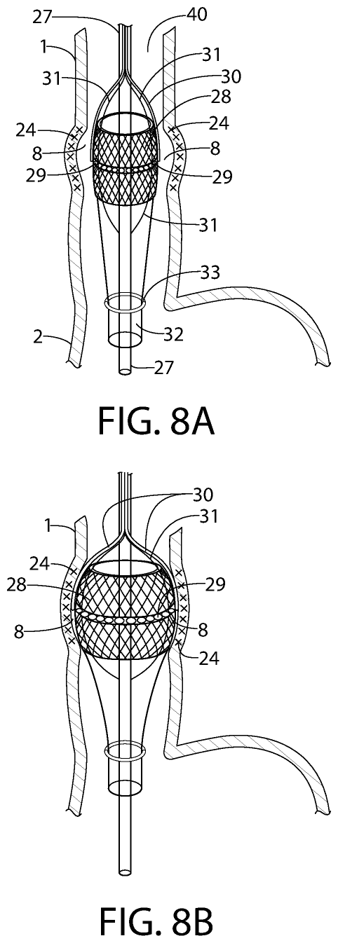

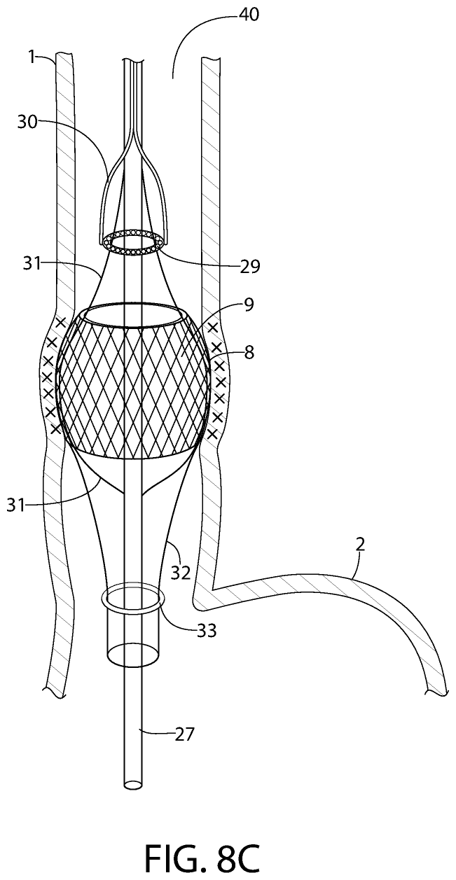

In FIG. 8A, the mesh ring (28) of the Therapeutic GARD™, here the GARD™ for GERD in its tubular form, is passed folded on the balloon (31) of a delivery catheter (27) through the mouth and into the esophagus and kept in place with a stretchable ring of magnetic beads (29). The tube of the GARD™ for GERD is also folded on the delivery catheter with a slip knot (33). The mesh is placed facing the bleeding niche (8) under endoscopic vision. Element 24 in FIG. 7 is the sites of the biopsies where viscous PRP has been sprayed. FIG. 8A shows the lumen (40) of the esophagus.

FIG. 8B illustrates the balloon (31) that is inflated and the mesh ring is held in position with the stretched ring of magnetic beads (29) that presses the mesh ring on the niche (8), where the mucosal biopsies have been made and the PRP with calcium gluconate have been added (see FIG. 7). The 2 strings holding the magnetic bead ring (30) are loose. The tubular part of the GARD for GERD device (32) is held folded on the delivery (27) with the slip knot.

As shown in FIG. 8C, the magnetic bead ring (29) has been pulled from the mesh ring (9) by pulling on the other end of the threads (30) at the head of the delivery catheter (not shown). This allows the inflated balloon (31) on the delivery catheter (27) to compress the mesh ring (9) on the niche with the coagulating blood and the added PRP with gluconate calcium (see FIG. 7). FIG. 8C shows the lumen (40) of the esophagus.

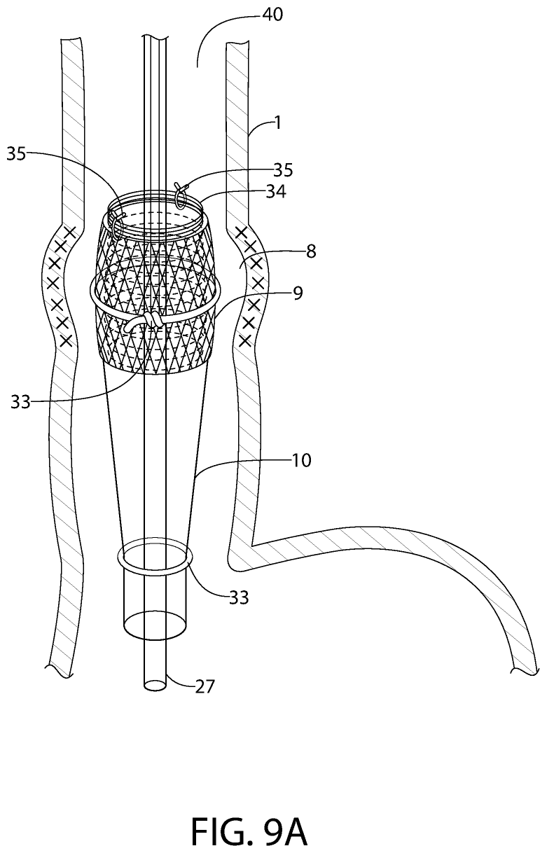

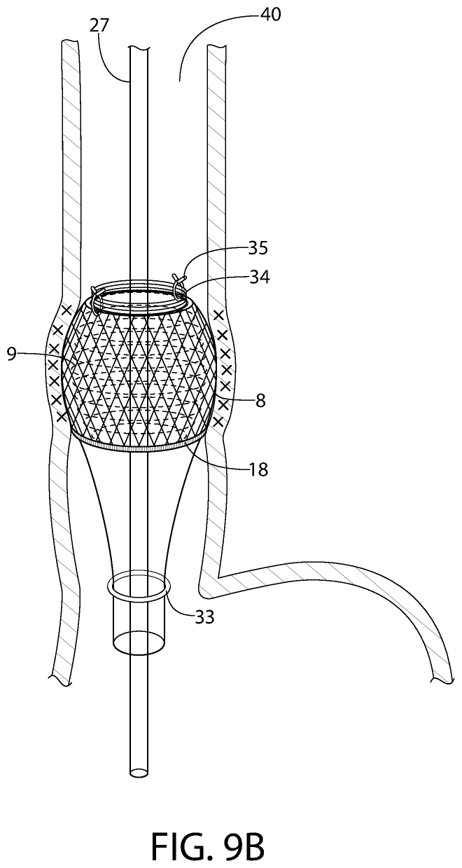

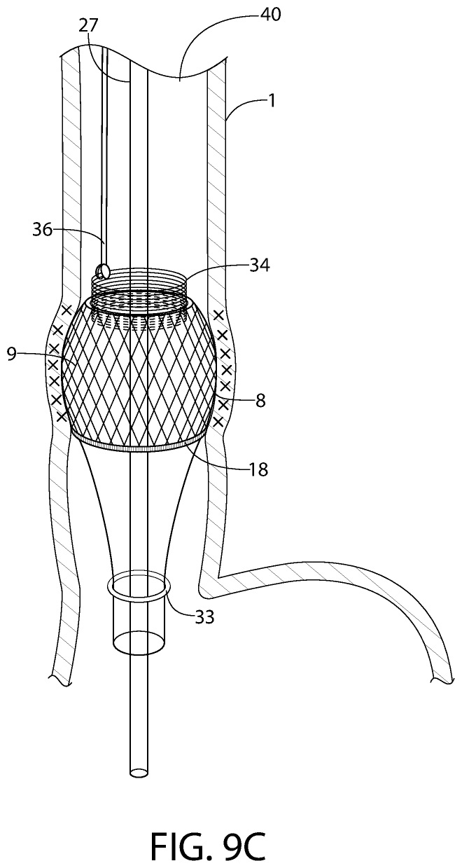

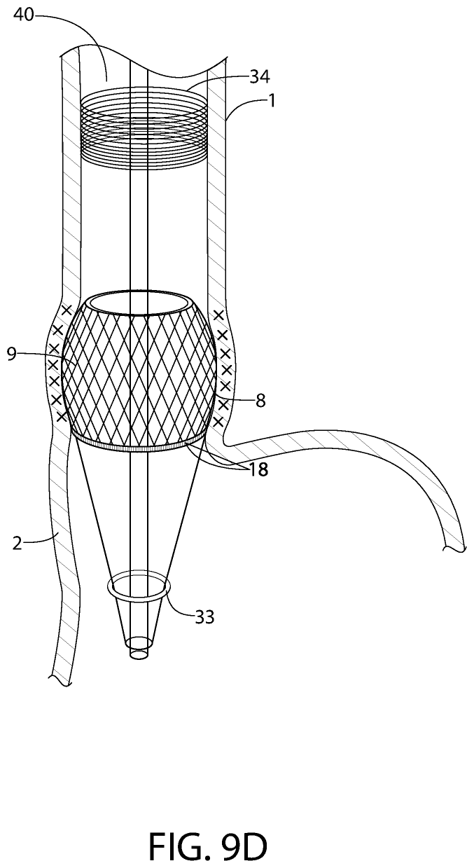

According to another embodiment of the presently disclosed technology, FIG. 9A illustrates a method of placement of the GARD™ for GERD on the niche (8) using a free helical pressure ring (34) similar to the ring of the DM-GARD (7) in FIG. 1 placed inside the mesh ring (9) that is folded and held In position on the delivery catheter (27) with a slip knot (33). When in position facing the bleeding niche (8), the slip knot is pulled. FIG. 9B shows the lumen (40) of the esophagus. The delivery catheter also has a balloon (31) placed on the delivery catheter.

FIG. 9B illustrates the GARD™ for GERD deployed with the helical pressure ring (34) exerting pressure on the mesh ring (9) on the niche (8). One or more knots (35) can hold the mesh ring (9) on the helical ring or spring (34) (e.g., the helical ring can contain or comprise a nitinol helical spring). Each knot (35) can be a slip knot that can be easily pulled out or a regular knot that have to be cut with a scissor through the endoscope (not shown). FIG. 9B shows the lumen (40) of the esophagus.

FIG. 9C illustrates that the knots (35) have been removed and the helical spring is gently pulled out with a forceps (36) leaving the mesh ring (9) positioned on the niche (8) where biopsies have been taken (x) and the coagulating blood with viscous PRP and calcium gluconate have been added (see FIG. 7). The slip knot (33) holding the tubular part of the GARD™ for GERD is still in position to exert a counterforce to the traction on the helical spring. FIG. 9C shows the lumen (40) of the esophagus.

FIG. 9D illustrates the mesh ring (9) is in position and the delivery catheter (27) is still kept in place holding the tubular valve with the slip knot. FIG. 9D shows the lumen (40) of the esophagus. The removable helical spring (34) has been pulled back towards the top of the esophagus to free access to the “inner” or luminal side of the mesh ring (9). The balloon (31) is not inflated and the slip knot (33) on the tubular part of the delivery is still in position.

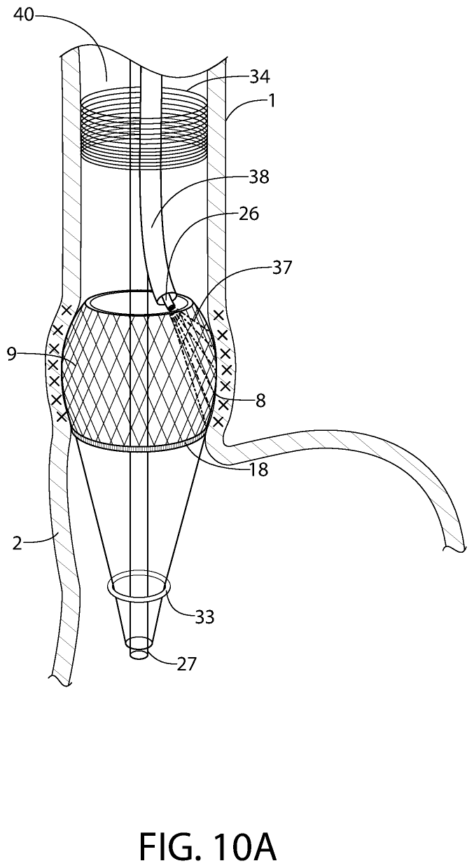

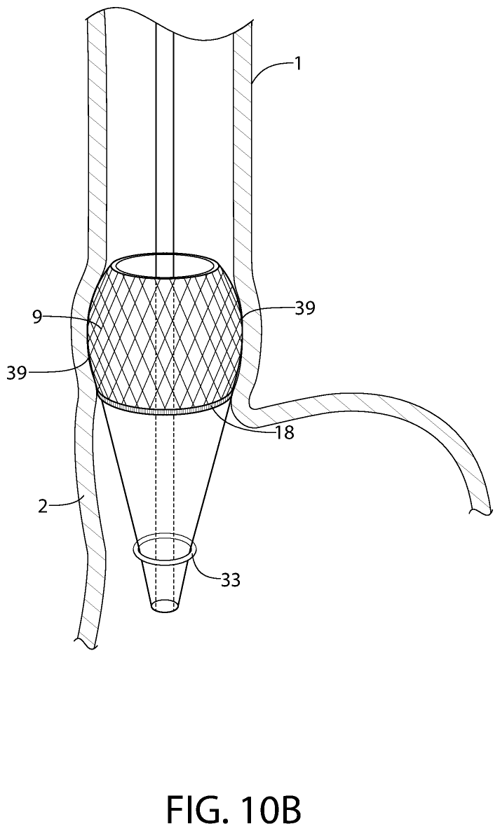

FIG. 10A illustrates that the mesh ring (9) is sprayed through the endoscope (38) with epithelial cells that have been placed in culture when the DM-GARD was first positioned in the esophagus and/or fragments of the biopsies of the esophageal wall containing adult stem cells of the epithelium of the esophageal wall cut up after biopsies were taken in the bottom of the niche (see FIG. 9) at the beginning of the procedure placing the Therapeutic GARD devices in the esophagus. In FIG. 10A, a balloon (31) that is fixed or otherwise secured on the delivery catheter is shown as not yet inflated.

The slip knot (33) of the tube is still in place and the delivery catheter (27) is in position. The helical spring (34) has been pulled upwards and the endoscope (38) passes through the helical spring to spray the luminal side of the mesh ring (9) with epithelial cells in PRP with calcium gluconate (37) to make the mixture more viscous and adhering to the mesh ring (9) so as to reconstitute the epithelial layer of the esophageal wall.

FIG. 10B illustrates the inflated balloon (31) on the delivery catheter pressing outwardly on the epithelial cells that were just sprayed on the mesh ring (9) so that the mesh ring (9) is “sandwiched” between the “external” niche containing coagulating blood and PRP/calcium gluconate and the “internal” reconstitution of the epithelial layer after spraying the adult stem cells in PRP to regenerate the epithelial layer in vivo.

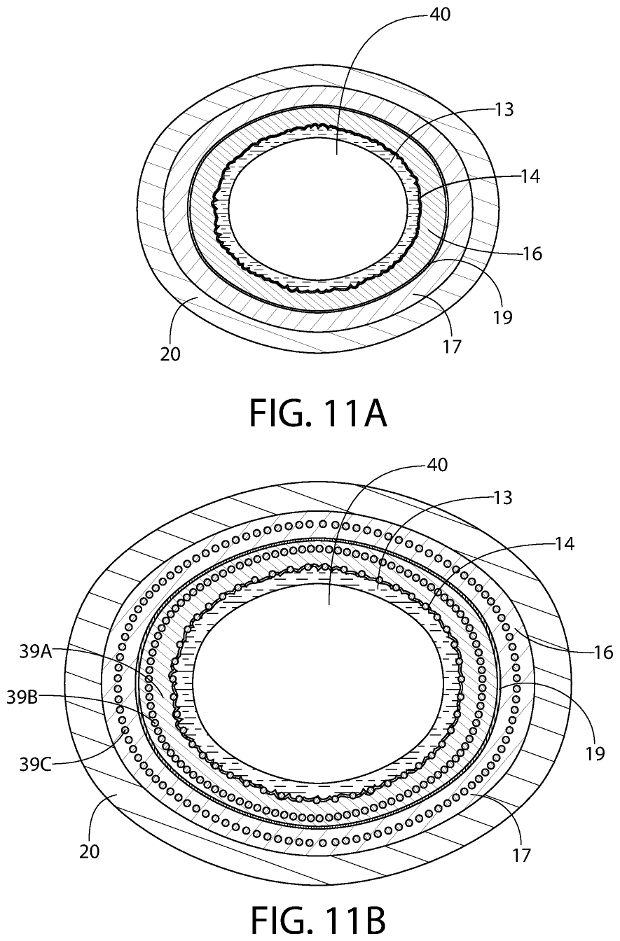

FIG. 11A illustrates a transverse view of the esophagus as shown in a longitudinal view in FIG. 6A. Visible in FIG. 11A is the esophageal epithelium (13), the basal epithelium (14), the lamina propria (16), the muscularis mucosae (19) separating the mucosal part (13, 14, 16) from the submucosa (17) and the muscularis propria (20). FIG. 11A shows the lumen (40) of the esophagus.

FIG. 11B illustrates various options for the depth of placement of the mesh ring in the esophageal wall at the end of the procedure. Location (39A) is the most superficial placement of the mesh ring at the level of the basal epithelium (Level A of FIG. 6). Location (39B) is the mid-level placement of the mesh ring in the lamina propria (16) of the mucosal wall (Level B of FIG. 6). Location (39C) is the deepest placement of the mesh ring in the submucosa (17) (Level C of FIG. 6).

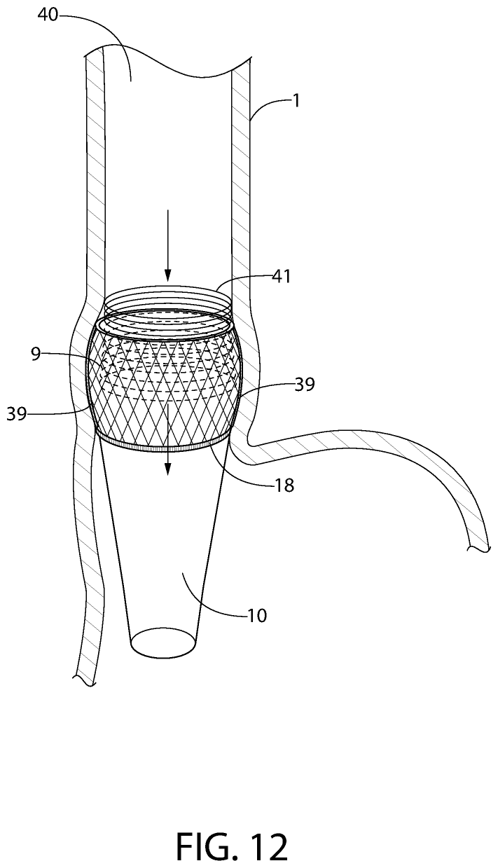

FIG. 12 illustrates a final compression ring (41) that is introduced to put pressure on the “reconstituted” esophageal wall after placement of the mesh ring (9) shown integrated in the esophageal wall (39) supporting the tubular or conical valve (10) of the Therapeutic GARDs™ within the esophageal lumen (40). FIG. 12 shows the esophagus (1) and the radio-opaque zone (18) of the Therapeutic GARD that allows location of the Therapeutic GARD after the compression ring with its nitinol radio-opaque springs is removed.

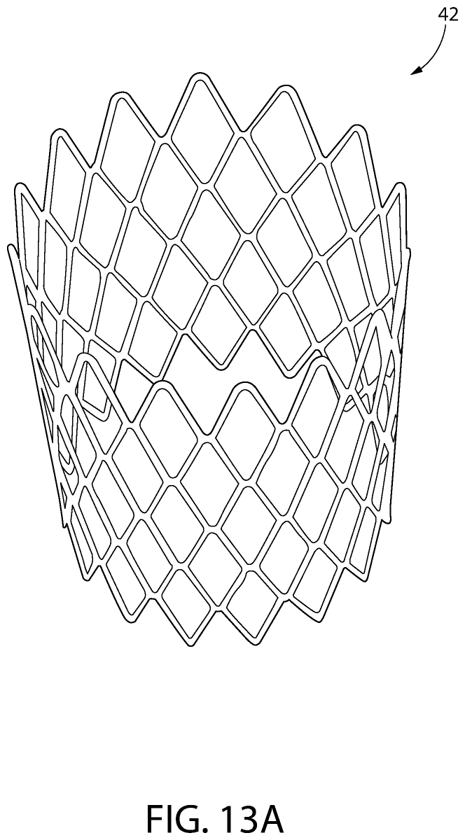

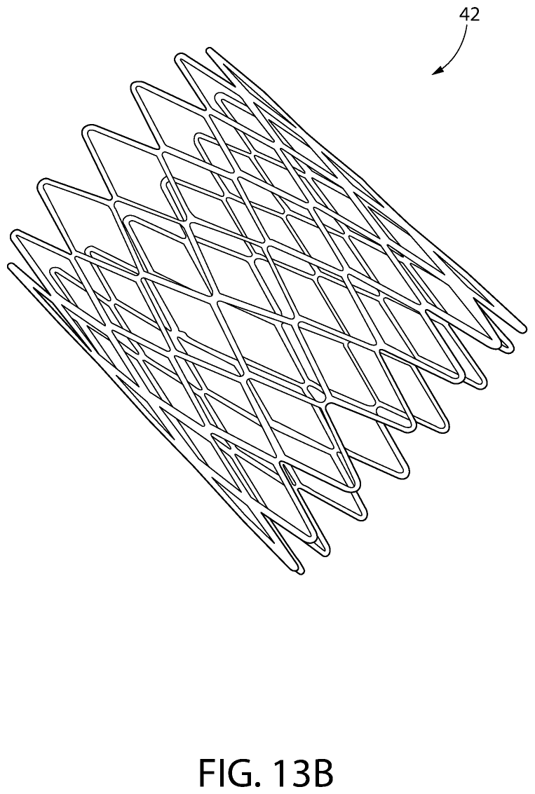

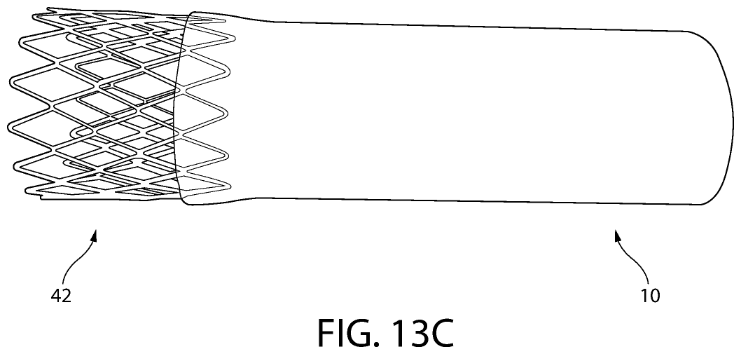

FIGS. 13A-13C show one embodiment of the presently disclosed technology that includes a circular ring 42, optionally formed of nitinol. In one embodiment, the ring 42 is molded in silicone and the DM GARD™ is put in place only temporarily to prepare the esophageal wall for placement of the Therapeutic GARD. In this embodiment, the ring 42 can optionally be removed after a predetermined period of time, such as but not limited to 1 to 4 weeks. In another embodiment of the presently disclosed technology, the ring 42 is not molded in silicone. As a result, in this embodiment, the ring 42 can be permanently integrated into by the esophageal wall.

An advantage of using the nitinol ring 42 is that when the healthcare professional pulls on the strings of the delivery catheter, the ring 42 springs into place. As a result, the ring 42 is placed on the bleeding site caused by the biopsies made just previously. This helps direct the adult stem cells of the esophagus that are injected in the Platelet rich plasma (PRP) solution on the lumenal side of the esophagus, thereby covering the nitinol ring 42 with cells that should reconstitute the normal internal wall (mucosa) of the esophagus. Other materials that have the same desirable properties as nitinol could be used to form the ring 42.

In another embodiment, the ring 42 can be formed of a polymeric material.

Until the presently disclosed technology was developed, healthcare professionals had not been able to place stents (nitinol or plastic) in the esophagus if there was no narrowing of the esophagus. The presently disclosed technology provides this benefit. In addition, previously healthcare professionals had not been able to hold a device in the lumen for therapeutic purposes, which the presently disclosed technology accomplishes.

Thus, in one embodiment the presently disclosed technology includes a combination of a niche created by the DM-GARD™ that is first placed in the esophagus and puts pressure on the wall of the esophagus creating a kind of bedding for the Therapeutic GARD™ (then the DM-GARD™ is removed). In addition, bleeding made by endoscopic biopsies and therefore coagulation after the bleeding helps hold the nitinol ring that support the anti-reflux and/or anti-obesity devices in the lumen of the esophagus and stomach. Furthermore, the presently disclosed technology can include putting the esophageal adult stem cells from the patient obtained by the biopsies in PRP was found to be a good milieu to put cells in culture or repair tissues (but not described to hold a foreign device like the Therapeutic GARD™ in position) and is used to reconstitute and/or repair) the internal mucosa of the esophagus in vivo after damaging it with the biopsies. The above allows for the integration of the stent in the esophageal wall at the mucosal-submucosal level of the esophageal wall and allows holding the anti-reflux and/or the anti-obesity devices in the lumen opening. This creates a completely new era of endoscopic treatment (without surgery) of these very common diseases on an ambulatory basis.

In one embodiment, injection of botulinum toxin will paralyze locally for a few weeks the peristaltic contraction and help the biological sealing of the nitinol ring that is thin (in the order of 0.3 mm thick) in the wall of the esophagus.

Optionally, the nitinol ring is thin, such as in the order of 0.3 mm think. When combined with the muscular layer, the two are about 3 mm thick, at most.

In one embodiment, a method of the presently disclosed technology can include first calibrating the diameter of the esophagus at the level of the esophagus (e.g., lower third) where the healthcare professional intends to put the Therapeutic GARD. Next, the healthcare professional can place the DM-1 GARD™ in and/or at the level identified above for approximately 1-2 weeks to create a niche (e.g., see FIG. 5). Eventually, an experienced healthcare professional could optionally skip this stage to avoid an additional endoscopy. Third, the method can include preparing the PRP from the patient's blood by spinning the blood twice. Fourth, the DM GARD™ is removed from the esophagus. Fifth, at least one and up to twelve biopsies can be taken from the bottom of the niche. For example, ten biopsies can be used for their stem cells and two biopsies can be used to have regular pathology to make sure the basal membrane is present as the adult esophageal stem cells are known to be right at the level of the basal membrane. This can be done for the control of the quality of the biopsies so they include stem cells.

The sixth step of the above-identified embodiment can include placing the biopsies (e.g., possibly cut-up in 2-3 pieces) in the PRP solution. Seventh, the nitinol stent of the Therapeutic GARD™ can be placed on the bleeding niche. Eighth, the PRP with esophageal adult stem cells can be injected on the internal (lumenal) side of the nitinol stent. Ninth, the PRP and stem cells can be compressed on the nitinol stent, such as with a balloon. An optional tenth step can include injecting botulinum toxin above the ring of the Therapeutic GARD™. An optional eleventh step can include positioning an additional helical spring ring on the nitinol stent for additional compression, such as for 1-2 weeks.

In another embodiment, a method of the presently disclosed technology can include, calibration. Second, the DM-1 device can be placed at the desired site for a predetermined period of time (e.g., 1 week), thereby creating a niche. Third, the DM-1 device can be removed, and biopsies can be made at the bottom of the niche to obtain adult stem cells. Four, the biopsies can be kept in the PRP obtained from the host (e.g., minipigs or patients). Fifth, the nitinol stent (DM-2 or Therapeutic GARD) can be placed on the bleeding site with the ring part in the bleeding niche. Optionally, lamellar devices can be used. Sixth, the PRP with the biopsies that include adult stem cells can be injected or sprayed on the luminal side of the ring to reconstitute the esophageal wall and incorporate the nitinol ring in the esophageal wall. Seventh, the DM-3 compression ring can be placed on the site for a predetermined amount of time (e.g., a week). Eight, the DM-3 ring can be removed after a predetermined amount of time (e.g., a week). Optionally, some PRP can be added and compressed with a balloon for a predetermined amount of time (e.g., 5-10 minutes). Once the balloon is removed, the procedure is finished.

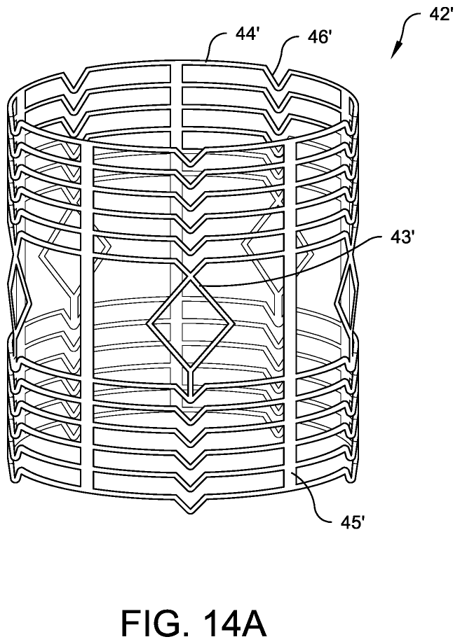

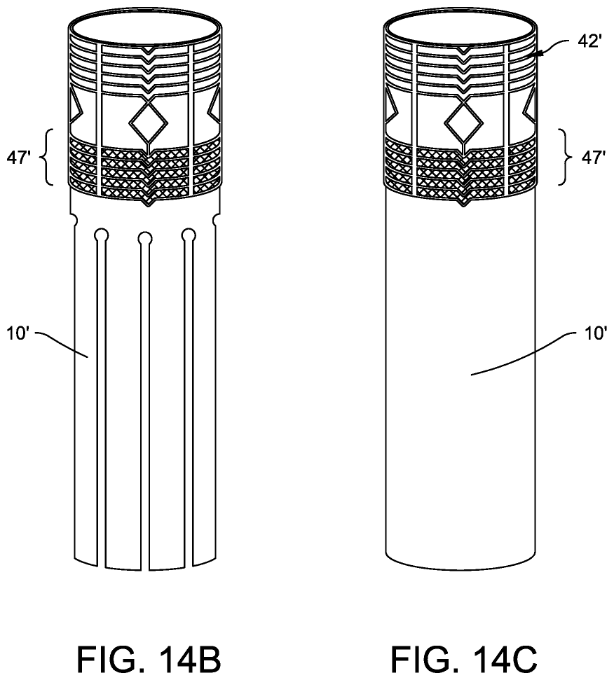

FIGS. 14A-14C show another embodiment of a medical device, stent, or ring 42′ of the presently disclosed technology. The stent 42′ of the present embodiment is substantially similar to stents described above. Like features between the embodiments are distinguished in the present embodiment by a prime (′) symbol. Certain notably differences are described below.

The stent 42′ of the present embodiment can include one or a plurality of spaced-apart connectors 43′. In one optional embodiment, each connector 43′ is in the form of a lozenge or diamond, and the stent 42′ includes five, equally spaced-apart connectors 43′ located at a vertical midpoint of the stent 42′. Of course, the stent 42′ can include more or fewer connectors 43′. Optionally, the top half of the stent 42′ is a mirror image of the lower half of the stent 42′.

Optionally, each connector 43′ contributes to allowing the stent 42′ to easily expand when starting in a collapsed configuration, or easily collapse when starting in an expanded configuration. This functionality can be useful, for example when the stent 42′ is introduced through the mouth of the patient and then expanded when released in position in the esophagus.

The stent 42′ can further optionally include a plurality of equally spaced-apart horizontal bars or supports 44′ and a plurality of equally spaced-apart vertical bars or supports 45′. In one embodiment, the horizontal bars 44′ extend perpendicularly to the vertical bars 45′. Each vertical support 45′ can extend from a bottom-most one of the horizontal supports 44′ to a top-most one of the horizontal supports 44′.

In one optional embodiment, the stent 42′ can include six horizontal supports 44′ above a row of connectors 43′ and six horizontal supports 44′ below the row of connectors 43′. In one optional embodiment, the stent 42′ can include five spaced-apart vertical supports 45′.

As shown in FIG. 14A, each horizontal support 44′ can include a plurality of spaced-apart grooves or bends 46′, optionally located in vertical alignment with one of the connectors 43′. The grooves or bends 46′ contribute to the stent 42′ being easily and/or selectively expanded, collapsed, and/or folded.

Optionally, any pair of vertical supports 45′ can be separated by a connector 43′, optionally located equidistantly therebetween. The connector part on top and bottom of the stent facilitates the attachment with knots to the ring 7′ that is soft with no spring in the ring 7′. The ring is made of 2 thin silicone rings, the external ring can have holes so the inflammatory cells creating a scar can pass easily through the connector for example 43′ in FIG. 14A then through the holes of the external layer of the ring 7′ but not through the internal layer that does not have holes

In one embodiment, such as shown in FIGS. 14B and 14C, at least a portion of the stent 42′ can be attached, such as by adhesive 47′, to at least a portion of a tube 10′, optionally of lamellar (FIG. 14B) or smooth (FIG. 14C) configuration. In particular, a lower portion, such as a lower third or quarter, can be glued to an upper portion of the tube 10′.

The stent 42′ is optionally formed of nitinol. In one optional embodiment, the stent 42′ is reconfigurable between an expanded configuration (e.g., for use in the patient) and a folded configuration (e.g., for removal from and/or insertion into the patient).

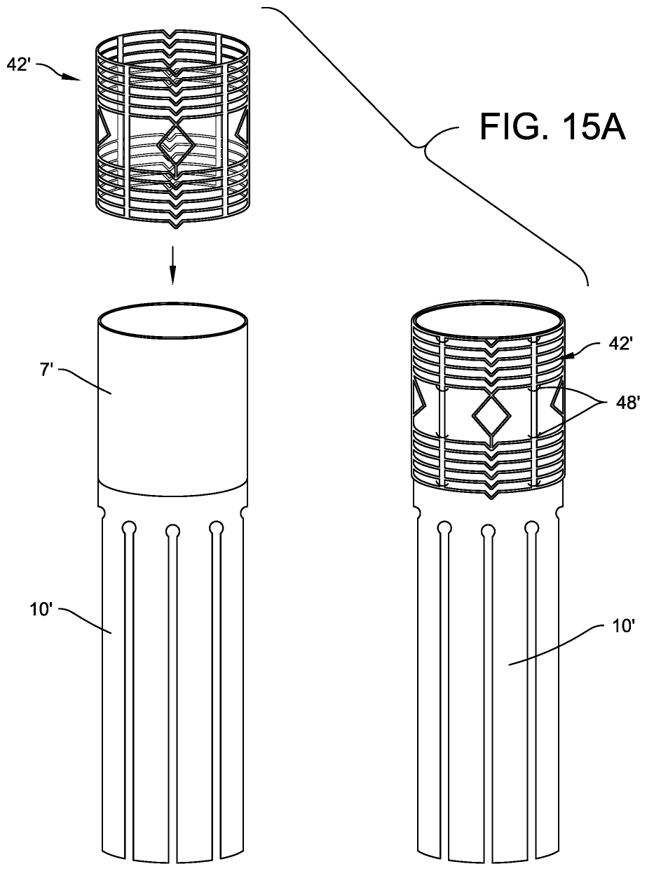

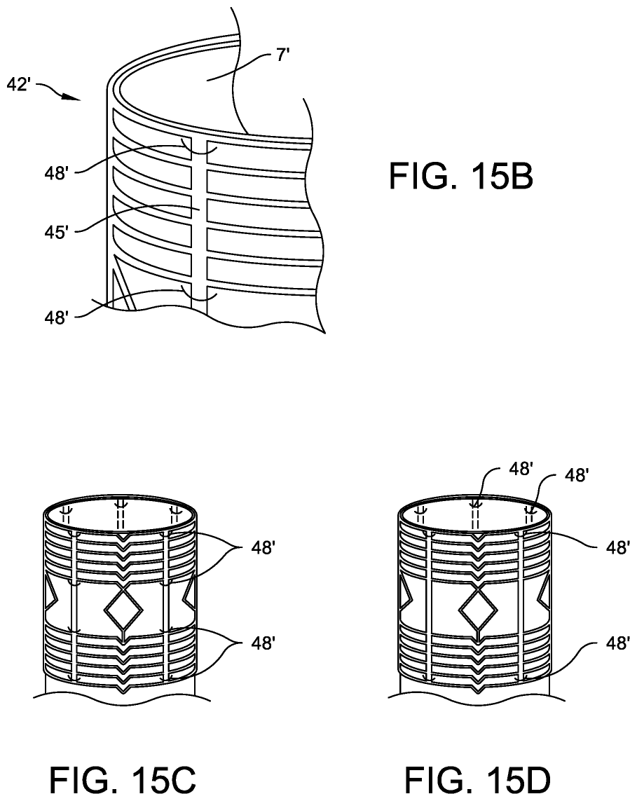

In another embodiment, such as that shown in FIGS. 15A-15D, the stent 42′ can be placed on its own in the wall of the patient's esophagus and can be linked or attached to a ring 7′ holding the active part (e.g., the DM2-GARD™ or DM-2). Optionally, the link or attachment can be one or more spaced-apart surgical sutures 48′ with knots or hooks.

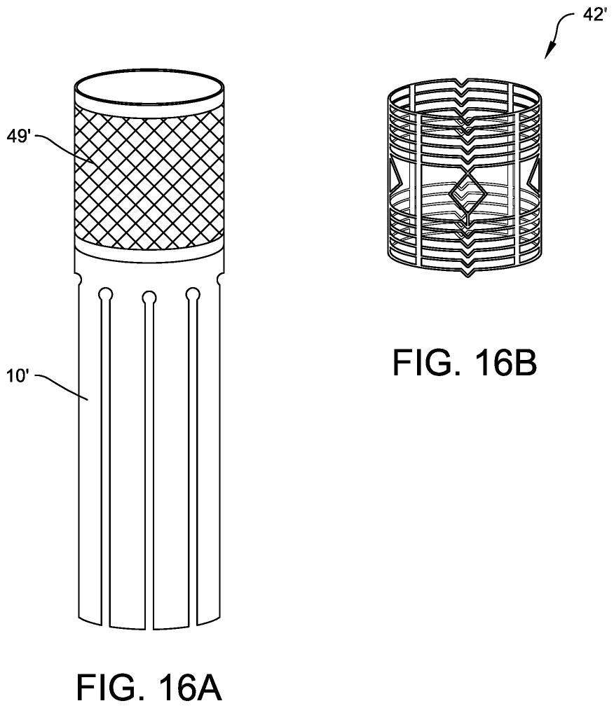

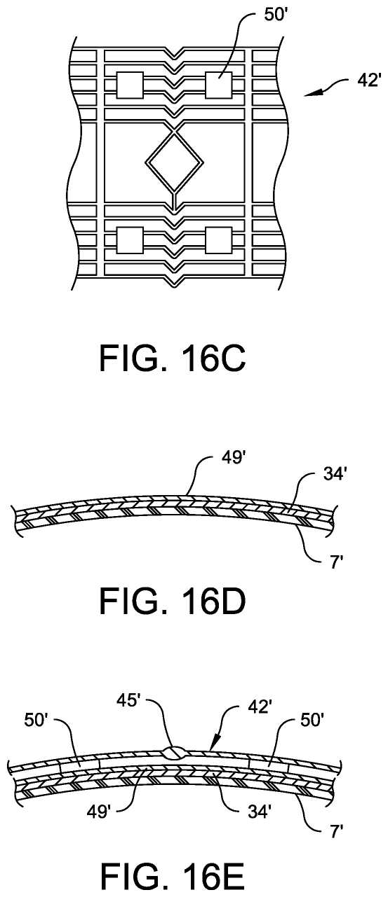

FIGS. 16A-16D show yet another embodiment of the presently disclosed technology, where at least a portion of the device is magnetized. In particular, at least a portion or an entirety of the stent 42′ is magnetized, which attracts the therapeutic parts of the ring holding the tube 10′ in addition to pressure exerted by the helical spring used with the DM1-GARD™.

For example, a ferromagnetic wrap 49′ (see FIG. 16A) can extend around the ring 7′. The stent 42′ shown in FIG. 16B can extend over the wrap 49′.

In operation of one or more of the above embodiments, after a preliminary (or preparatory endoscopy) where balloon calibration of the esophagus is done to determine the size of the esophagus above a predetermined line, which can be at the mucosal border between the esophagus and the stomach, and before any biopsies are taken, then a regular endoscopy with a prior basic blood work-up that includes at the very least a complete blood count, standard coagulation studies (CBC with platelets and INR), basic liver and kidney functions can be performed before standard intravenous sedation is done (with Propofol or Flurazepam) to determine if there is any reflux esophagitis present then biopsied to rule out Barrett's esophagus, a well-known precancerous condition always checked by endoscopists, eosinophilic esophagitis, a less frequent allergic related esophagitis, any esophageal or other lesions.

If the patient has chronic esophagitis and responds to proton pump inhibitors (e.g., omeprazole, Nexium, Dexilant, Pantoprazole, etc.), but does not want to continue the medicine forever and if the symptoms of heartburn recur after stopping the PPIs and does not want to consider surgery (now mainly the laparoscopic Nissen operation or variations with an incomplete fundoplication, or any another endoscopic method developed for GERD or obesity), the endoscopist can optionally order the appropriate size kit to place the GARD™ with the versions that will reach the market.

If the patient does not have a classical reflux disease symptomatically but has esophageal complaints that do not respond well to PPIs, then the patient may need an additional work-up that can also be done for standard GERD, with preferably a 48 to 96 hours telemetric pH study with a small capsule placed in the esophagus to prove reflux (called the Bravo capsule), as well as esophageal manometry, or an esophageal impedance test that can replace the telemetric pH metric studies. If GERD is demonstrated and no other disease is diagnosed during this work-up, then placing the GARD is certainly a valid option.

In the embodiment shown in FIGS. 14A-C, the stent 42′ can optionally include silicon covering or located on the lower part of the stent 42′. Optionally, about or exactly 25% or 30% of the total length of the stent 42′ can be covered in silicon to glue the lamellar tube to the stent. For example, about or exactly 70% or 75% of the upper portion of the stent 42′ is not covered by silicon, but the lower (e.g., optionally six) horizontal bars 44′ can be covered with the silicone of the tube 10′. Of course, the number of horizontal bars 44′ can covered with silicon can vary, with possibly fewer bars or more bars depending on clinical experience.

For Obesity, the tube 10′ will be longer than for non-obesity application, even much longer to go to the stomach, and if possible cross the stomach (mimicking a “sleeve gastrectomy” operation for obesity), but without surgery and reach the duodenum and into the jejunum to mimic a gastric by-pass operation. As a result, the stent 42′ might be a little longer (e.g., at least 30 mm long instead of 25 mm presently) and at least 10 mm covered with silicone to hold the tubes 10′ in place than in non-obesity applications.

Referring again to FIG. 15A, the stent 42′ has approximately the same size as the ring 7′ or DM1 that has been removed and the entire stent 42′ can be placed in the niche. This is unlike one optional use of the embodiment of FIGS. 14A-C, in which optionally only the top % of the stent 42′ is placed in the niche. The Therapeutic GARD™ of FIG. 15A can optionally include a silicone ring 7′ with a lamellae or tubular tube 10′ that is placed within the ring 7′, except without the helical spring 34′ (see FIG. 16D) (sometimes referred to as the DM2), and is attached to the stent 42′ with knots 48′, for example, using surgical suture thread. In one version of the present embodiment, the silicone ring 7′ does not have a helical nitinol spring within the ring as the DM1-GARD™ has.

A difference between one use of the embodiment of FIGS. 14A-C and the embodiments of FIG. 15A-D is that in the former glue, such as silicone glue, can be used to bind the GARD to the sent 42′ or blood can be left to coagulate spontaneously. In the latter, knots can be placed between the stent and the DM2.

In the embodiments of FIG. 15A-D, the knots 48′ can optionally be placed from, at, or around the vertical bars 45′ toward the lumen of the DM2 where food passes from the mouth to the stomach. For that reason, in one embodiment, the knots 48′ are placed on the stent 42′ side (e.g., the outer side).

As shown in FIGS. 15C and 15D, the thread used to form the knots 48′ is shown passing through the wall of the endoscopy, particularly if the threads are black on a light silicone background. Optionally, hooks could be an alternative to sutures, but hooks can be more completed to remove, if necessary, than cutting sutures.

Optionally, the presently disclosed technology can include twenty spaced-apart knots 48′ is a 5×4 array (see, e.g., FIG. 15C). In another arrangement, the presently disclosed technology can include ten spaced-apart knots 48′ in a 5×2 array (see, e.g., FIG. 15D). Other arrangements are also possible, including greater or fewer total number of knots, in different array configurations.

Optionally, ten spaced-apart knots would in principle decrease infection risk and favor a microcosm pocket of reparatory cells between the DM2 and the stent 42′ in the wall of the esophagus. Without the helical spring inside the ring 7′, it is possible to fold the DM2 part of the Therapeutic GARD™ attached to the stent 42′ and fold it on the delivery held on the delivery catheter with the escape knots. If the helical spring 34′ is used as in the DM1-GARD™ with the hard helical spring inside, it can be more difficult to fold the stent and the ring with the helical spring tightly enough because the two rings are too strong to be kept folded tightly on the delivery catheter to be passed through the mouth and throat (pharynx) and upper esophagus through the strong upper esophageal sphincter without risking to hurt the patient or risk deploying the devices prematurely before the lower esophagus is reached where they should be placed. This situation could have very challenging if the patient's airways are obstructed.

Optionally, the silicon ring 7′ will not put much pressure on the stent 42′, but should efficiently prevent the inflammatory cells trying to penetrate the esophageal lumen from doing so and retain the inflammatory cells in several cavities between the stent and the back of the silicone ring.

A main procedure difference of the embodiment of FIG. 15A is that since the stent 42′ and the DM2 are placed together, the healthcare professional selects to spray the fibrin glue, it should be done immediately after the biopsies of the niche is done, and before both the stent 42′ and the DM2 are placed together. Once in place, a DM3 device can be placed within the ring 7′ for at least 1 month, but then would need to be removed at a third endoscopy, which would be better if it could be avoided. So, in certain applications, an advantage of the embodiment of FIG. 15A over the embodiment of FIGS. 14A-C is that a third endoscopy might be potentially avoided with the embodiment of FIG. 15A.

Optionally, to avoid or limit the risk of infection from threads between the DM2 and the stent 42′, antibiotics can be injected at the time the Therapeutic GARD™ is installed.

Referring to the embodiments of FIGS. 16A-E, an advantage of a magnetic link or connection between the DM2 and the stent 42′ is that the lack of a physical connection could decrease an invention risk. This is because even if minute particles, e.g., of food, could penetrate or enter between the DM2 and the stent 42′, the particles would be flushed with white blood cells and mononuclear cells (e.g., lymphocytes, monocytes, macrophages) specialized in fighting any infection.

In the case of a magnetic connection between the DM2 and the stent 42′ holding the DM2 part in place, both the stent 42′ and the DM2 will need one or more relatively strong magnets. In one optional embodiment, the magnetic part could be the vertical bars 45′ of the stent 42′, or the entire stent 42′ could be magnetic, but should still retain its elasticity to be introduce in the through the mouth into the esophagus.

The DM2 can be made magnetic with different options. For example, one option is to have multiple, spaced-apart, and relatively small magnets 50′ on or in the stent 42′. For example, the magnets 50′ can be anywhere between 1 mm and 10 mm in diameter, but optionally 3 mm to 5 mm in diameter, and optionally can be attached (e.g., via glue) on the top and bottom vertical bars 45′ and/or the horizontal bars 44′, for example, of the stent 42′. Optionally, the magnets 50′ can be located on the inside of the stent 42′ and placed in the bleeding niche as described above. The magnets can have any of a variety of shapes, such as circular or square.

In one embodiment, such as that shown in FIG. 16B, the ferromagnetic sheet 49′ can be attached (e.g., via glue) to the outside of the DM2 ring optionally with a helical spring in the ring as mentioned above. The silicone ring of the DM2 supporting the tube 10′ can have a diameter of 1 mm to 2 mm less than the internal stent diameter to facilitate placement of the stent and pass the folded DM2 device described earlier through the stent 42′ with the magnets facing inwards.

In operation of one embodiment, the DM2 can be placed exactly at the level of the stent and released by pulling on the slip knots so that the outside part of the DM2 ring with the magnetic sensitive ferromagnetic ring faces with the endoscopically and give access to the inflammatory cells around the ring through the stent around the ring (see FIG. 16D). If a magnetic connection is used, it would allow replacement of the device by pulling on the DM2 with so-called rat tooth or crocodile tooth forceps sold on the professional endoscopy market by the Olympus company and other companies selling endoscopic appliances.

In a similar but opposite design, the magnet(s) 50′ can be placed outside of the stent 42′ (instead of inside as described here above) that is on the side facing the esophageal wall. This makes it easier to glue or otherwise attach the magnet(s) 50′ on the stent 42′ from the exterior of the stent 42′. Even if the magnets are anywhere between 1 mm to 5 mm thick, this thickness does not let the ring of the DM2 device have an immediate contact to the interior of the stent 42′ because of the “bump” created with the magnet. Optionally, the thread of the magnet is only 0.3 mm thick in one embodiment.

Optionally, each magnet can be about 2 mm to 8 mm in diameter and 2-3 mm in thickness, and can be glued or otherwise to the upper and/or lower parts inside the stent 42′.

In one embodiment, such as that shown in FIG. 16C, the presently disclosed technology can include four small neodymium or other magnet composition (e.g., rare earth, etc.), two on top and two on the bottom part of each of the five areas between the five vertical bars 45′. Optionally, the magnets 50′ are placed in such a way that it is possible to fold the magnet and place the magnet bearing stent on a delivery catheter. and introduce the stent and delivery in the patient's esophagus as described previously.

The cross section shown in FIG. 16D shows the silicon ring with the ferromagnetic layer of the DM2-GARD™. In FIG. 16E, the stent 42′ and magnets 50′ were added to that shown in FIG. 16D.

It is possible to calculate the physical forces involved with magnets 50′ depending on their size and compositions and make prototypes to determine if the strength between the stent 42′ and any DM-GARD, the DM1 GARD™, or the DM2 GARD™, and whether the stent 42′ would be strong enough to help hold the device in place.

If the stent 42′ is placed in the mucosa-submucosa area of the esophageal wall, as described above, the infectious and immune cells will rapidly gather locally around the stent 42′ and try to push the stent 42′ with the help of the muscular layers of the esophagus (muscularis mucosae, circular internal muscular layer). This will try to compress the stent 42′ into the lumen of the esophagus and longitudinal external muscular layer that will attempt to push the stent 42′ into the stomach (peristaltic wave). The pressure on the stent 42′ to be pushed into the esophageal lumen, this pressure would be opposed by the counter-pressure created by the DM2 device with its fairly strong helical spring in this case very similar to the DM1 device. So, the DM2-GARD™ can counter-act the pressure on the stent 42′ if the pressures are more or less equivalent and help keep both the stent 42′ in the slayer of the esophageal wall (mucosa-submucosa) and the DM2-GARD™ in the lumen. With time if both devices cannot be eliminated and fall into the stomach, it is reasonable to believe that scar tissue will develop and hold the DM2-GARD™ in place.

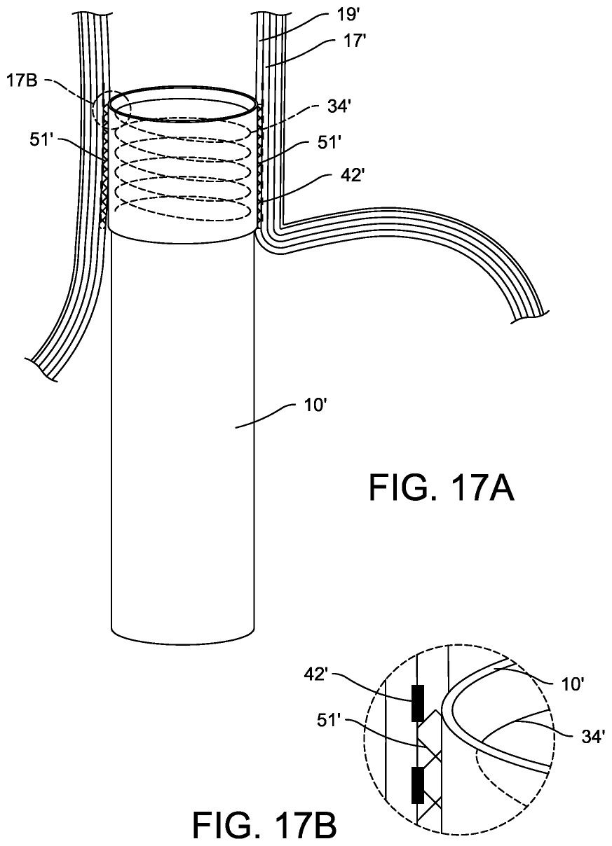

In FIG. 17A, the stent 42 is seen at the limit of the mucosa-submucosa, more or less at the lever of the muscularis mucosae. In one embodiment, the DM1-GARD™ penetrates for at most 1 mm into the mucosa of the esophagus and creates a niche. Optionally, after 1-2 weeks, the DM1-GARD™ can be pulled out and biopsies are taken of the niche and the stent 42′ can be placed using a delivery system. Optionally, fibrin glue can be sprayed on the stent 42′, then the DM2-GARD™ can placed in the lumen as shown in FIG. 17A.

In FIG. 17A, a tubular DM2-GARD™ (instead of a Lamellar active part of the device as shown in all the preceding versions) and a stomach that has been operated with a sleeve-gastrectomy for obesity is shown. The fundus is very small in comparison to a normal gastric fundus. It is known that at least 15% of the patients that are operated for obesity with the sleeve gastrectomy have very severe reflux, even some patients who did not have reflux before surgery despite their obesity which is a risk factor for GERD. Gastric bypass patients lose between 50 to 80 percent of excess bodyweight within 12 to 18 months, on average. Gastric sleeve patients lose between 60 and 70 percent of their excess body weight within 12 to 18 months, on average.

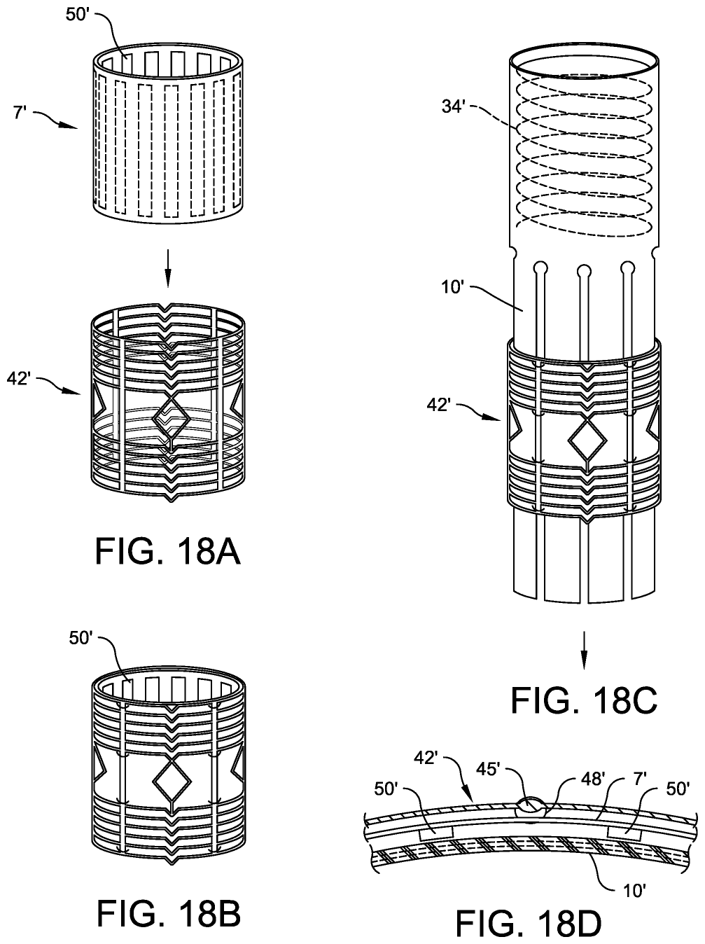

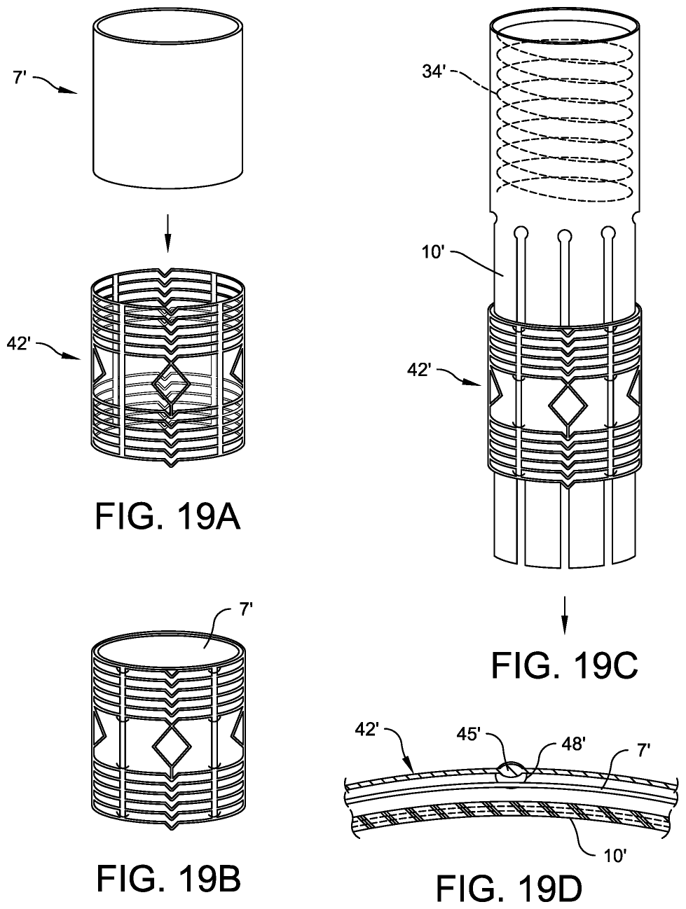

Referring to FIGS. 18A and 19A, the ring 7′, such as an intermediary silicon ring, can be placed into the stent 42′, and then the combined ring 7′ and stent 42′ can be placed together in the esophagus. As shown in FIG. 18A, the ring 7′ can optionally include one or more of a plurality of spaced-apart magnets 50′ therein or thereon, which can be attracted to the magnets 50′ of or on the stent 42′. As shown in FIG. 19A, the magnets 50′ can be omitted from the ring 7′.

Once the combined ring 7′ and stent 42′ are within the esophagus, the device to the right in FIGS. 18A and 19B is placed in the combined ring 7′ and stent 42′. The stent 42′ with the helicoidal ring 34′ stays in position because of the size, or diameter, of the rings.

In FIG. 19A, the stent 42′ can have a diameter of approximately or exactly 27 mm. The ring in FIG. 19A can have a diameter of approximately or exactly 26 mm, and can be placed within the stent 42′ during manufacturing and the knots are tied. This part of the device is placed on the first delivery catheter and liberated within the esophagus. The ring optionally deploys because it is pulled by the stent to which it is attached with knots. Optionally, one or two balloons can be employed to install the device shown to the right in FIG. 19A. When inflated, the balloon(s) will help the device deploy as the ring is optionally quite soft and elastic, made only with silicone without a spring, but can still be removed and replaced, if necessary.

The following optional operation of the first endoscopy can be utilized by one or more of the embodiments described above. The appropriate size DM1-GARD™ can be placed in the esophagus using a delivery system for approximately 2 weeks. Optionally, the symptoms of the patient should be assessed (e.g., a GERD score used before and after placement can be used) after one week to determine if there is improvement and a second pH metric study over 24-48 hours can be part of the clinical trials, but could also be used in Refractory GERD patients (that is, patients not responding or responding badly to PPIs that are frequent and are often referred to specialized Gastroenterologists/Endoscopist that are faced with no good option and the GARD method).

In one embodiment, a unique advantage with the GARD™ is that the DM1-GARD™ should not go beyond 4 weeks (optionally 2 to 4 weeks) that will let the healthcare professional know if the GARD method is helpful to the patient or not. This feature is unique as no other surgical or endoscopic method offers the possibility to test the technique in a given patient before moving on to a definitive phase.

After 2-4 weeks, the DM1 can be pulled out very easily with standard endoscopic forceps. If the device helped the patients, the endoscopist can place the final stent by making about 20 biopsies with the standard biopsy forceps around the niche made by the DM-GARD™, release the stent with the lamellar anti-reflux device in the niche covered with blood so that the stent is lodged in the niche and the lamellar tube below closer to the stomach (distally to the mouth) and spray the niche with adhesive (e.g., fibrin glue) to fixate the stent in the niche or just let the blood coagulate normally without using fibrin glue. Before starting this phase, the endoscopist can optionally have chosen between two different stents—either the stent with the lamellar silicone tube glued to the stent as described above (e.g., FIGS. 14, 16, and/or 17) or the independent stent (e.g., FIG. 15) with a silicone ring without a spring within the ring that has knots attaching the silicone ring with the lamellar tube to the stent. The ring and lamellar tube are similar to the DM1 device but without a helical spring. The ring of the Therapeutic device of FIG. 15 is attached with surgical sutures to the stent.

Optionally, in each of the embodiments shown in FIGS. 14-17, the normal regenerative esophageal cells will try to expel the stent from the esophagus wall into the lumen of the esophagus as they do for any foreign object in the following days, weeks and months following implantation. In the upper and lower parts of the stent that is identical in FIGS. 14 and 15, the regenerative cells should have more difficulty to pass through the stent in the upper and lower part because there is less space between the horizontal nitinol little bars of the stent. This is particularly true in the embodiment of FIG. 14, as the lower part of the horizontal nitinol bars are covered by the silicone used in the intermediate part of the device, that is the part where the silicone is glued to the stent where no cells should be able to pass from “outside” the stent to “inside” the stent or from the wall of the esophagus where the stent is implanted towards the lumen of the esophagus. On the other hand, in the middle of the stent where the lozenges are located, there is lots of space and the regenerative cells should favor this route and in fact pass on the inside of the stent and envelope the stent with neutrophils (one of the main types of white cells that defend the body from infections.

Normally, if a foreign body cannot be expelled of the body as is expected to happen with the design of the stent described herein, it will then be surrounded by monocytes and local macrophages that will attempt to destroy, literally eating fragments of the foreign material. In one embodiment of the presently disclosed technology, the nitinol is quite elastic, but is made of a very resistant material that should not be affected by the monocytes and macrophages nor should the silicone. Therefore, one can expect that the stent will stay locally in the esophageal mucosa “wrapped” by inflammatory cells and eventually develop a small then larger quantity of local scar tissue 51′ that is strong and help the stent stay within the esophageal wall with the silicone device glued to it indefinitely and therefore block reflux with the lamellae that are in the esophageal lumen that let food pass but at low reflux pressure block the reflux and at high reflux pressure as in vomiting will fold back up to let the body expel the food through the mouth. As one usually drinks water after vomiting to rinse one's mouths and esophagus the water will help the lamellae resume their original position. A tubular device is much more efficient in stopping reflux but will probably resist vomiting forces and vomiting risks tearing the device. In some cases, such as after a sleeve gastrectomy for obesity, the second most frequent obesity operation after a gastric by-pass, % of the stomach has been removed and the vomiting forces should be much weaker. As these patients often have severe reflux after the operation, a tubular valve could be often indicated. This can be an important point in obesity surgery as the “sleeve” operation is much easier to perform in very obese patients than the gastric by-pass and has much less complications except severe reflux in a number of patients who have to be reoperated to change their sleeve gastrectomy into a gastric by-pass. These operations in very obese patients have at least a 1% mortality that could be prevented by using the GARD™ technique.

In one embodiment, once the stent with glued lamellae is in position, to avoid having the regenerative cells that will normally invade the stent as described enter the esophagus and risk creating a narrowing (stenosis) of the esophagus which is a known risk, a small device that includes only the ring of the DM1-GARD™, optionally called the DM3, can be placed for a few months (e.g., 1 to 6 months) on the esophageal area of the stent to prevent any stenosis and help fixate the stent in the wall of the esophagus until scarring around the stent occurs.

With respect to the embodiments of FIGS. 14 and 15, optionally the Therapeutic GARD™ device can be safely and efficiently in place between 3 to years with a lamellar device described previously, if with time the lamellae are covered with food or attacked by acid or both, it will be fairly easy to cut off the lamellae at endoscopy and replace this first device with another new device will be available to replace the device. This approach will allow then to chose different devices of the same family either for GERD by changing the number of lamellae, between 4 and 20, and optionally between 4 and 12 with the more lamellae present decreasing the risk of displacement during vomiting but normally also decreasing in theory the efficacy of reflux protection (this point has yet to be proved in vivo). The replacement of the original device can be done in only one endoscopy. Options to treat overweight patients and obesity will also be available over time once the stent holding the devices in the wall of the esophagus is well in place and blocked with natural scarring tissues, similarly to coronary or vascular stent although the situation in the esophagus (as in the rest of the gastrointestinal tract) is very different from vascular mucosa as the mucosa is different and behaves differently in vascular tissues (with an endothelium) than in the gastrointestinal tract.

One achievement of the technique of one embodiment of the presently disclosed technology is to put a platform in place in the esophageal wall and esophageal lumen where different devices (e.g., number of lamellae of the lamellar tube, length of the tubular valve stopping or potentially crossing the stomach) can be employed and then depending on the length could mimic the sleeve gastrectomy with a tube crossing the stomach or ending right before the pylorus or if with time a longer tube can be used and a peristaltic technique of the tube installed in the tube. A device that could mimic the sleeve gastrectomy with its technique of restriction (e.g., the tube) and malabsorption (e.g., the food in the tube cannot touch the mucosa or be in contact with bile, pancreatic secretion or duodenal/small bowel secretions that are absolutely necessary to allow food absorption). This technique would then allow important weight loss as needed in more severe type 2 or 3 obesity that is morbid obesity, all without surgery and without scars, all through the mouth and potentially with experience a same day procedure.

In the embodiments of FIGS. 18 and 19, the active device (i.e., the combination shown in FIGS. 18C and 19C once the combination of the sent and the ring are moved to the top of the tub) can be replaced by pulling out the device. So, removing the devices and replacing them should be easier than at least some of the other embodiments.

Another option would be to first place a stent with magnets inside the stent, facing the lumen of the esophagus as shown in FIG. 16C in the bleeding niche with a first delivery catheter, then place a Therapeutic device (this method avoids a third device DM2 as described in FIG. 18A and FIG. 18C with the vertical magnets as described in FIG. 18A or the ferromagnetic sheet described in FIG. 16A using the method described in FIG. 18C).

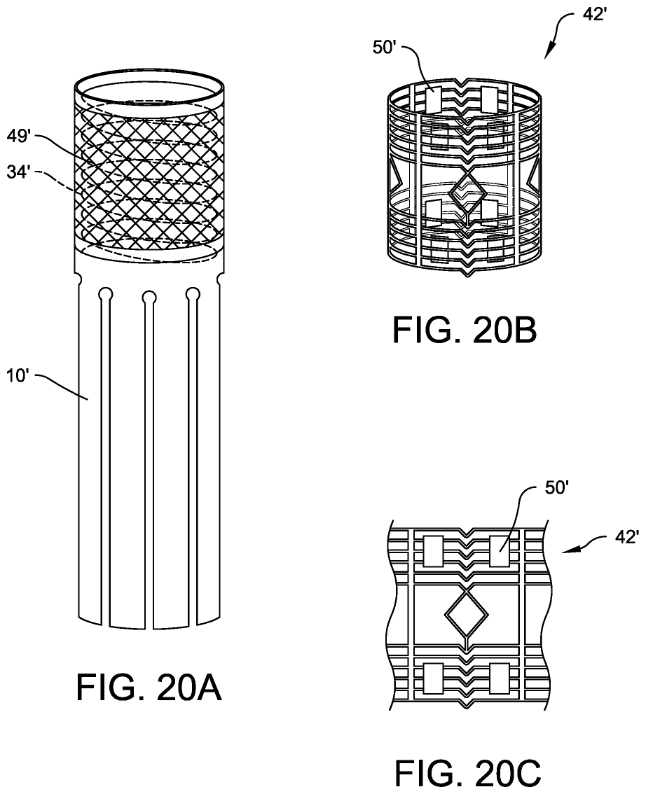

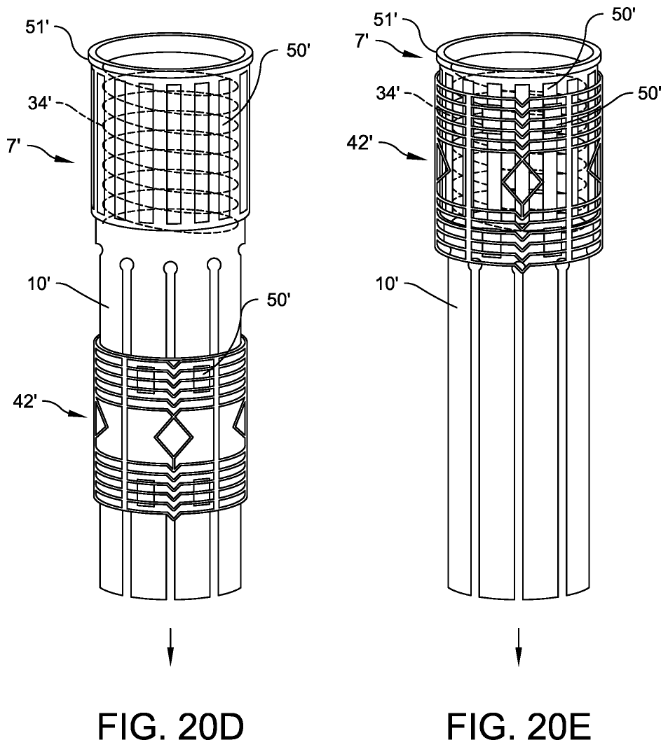

FIGS. 20A-20E show an embodiment with a plurality of spaced-apart magnets 50′ on an inside of the stent 42′. By using this method showed in FIGS. 20A-20E, an advantage of placing the stent in one procedure with the magnets to reinforce the contact between the ring of the Therapeutic Device placed during the same procedure with a second delivery catheter. To prevent having food pass between the Therapeutic magnetized device and the stent, a rim could be placed at the top of the Therapeutic GARD device with a thickness corresponding to the addition of the thickness of the magnets inside the stent (e.g., about 1-2 mm) and the thickness of the ferromagnetic sheet or vertical magnetic bars shown on the top figure of FIG. 18A. With this method, the following requirements would be fulfilled:
a) have only 2 endoscopies, the first one placing the DM1 Diagnosis GARD to determine if a long-term device is helpful to the patient objectively and subjectively as described earlier. Then place the stent with magnets and the Therapeutic GARD during a second procedure.
b) using this technique for Therapeutic GARDs or Obesity GARDs
c) replacing the Therapeutic devices when and if needed with the same basic design in the new Therapeutic device or adapted to any changes the patient has had to his/her symptoms, as doctors would do by changing the dosages of any medication the patient is taking if the symptoms improve or worsen and then a gastroscopy is performed to confirm that the change of symptoms correlate to a change of endoscopic pathology.
d) with a circular rim decrease the risk that food can pass between the stent and the Therapeutic GARD.

Other applications of the techniques of the presently disclosed technology can include placing the stent 42′ in a desired location for monitoring the heart, which can be just in front of the esophagus such as for a long-term term transthoracic echocardiogram. This and other applications in cardiology are possible, as well as in food monitoring, lung monitoring, body temperature variation monitoring, as well as ultrasound monitoring of the chest and/or spine.

Optionally, the stent 42′ can form part of supported platform concept in other parts of the gastrointestinal (GI) tract, in particular in the colon or in the duodenum to monitor suspicious lesions in the pancreas, such as some pancreatic cysts that could develop into cancer with time, particularly if new blood monitoring techniques based on detecting some known mutated circulating cell-free DNA associated with cancers or pre-cancerous lesions now in development, some focused on the pancreas where lesions are often detected very late to be cured continue to develop very rapidly as well as in the lung where cancers are often detected quite late. The stent 42′ could of course be used in the esophagus, which is why the first endoscopy to rule-out any precancerous lesions, such as Barrett's, is important. So even if one embodiment of the present platform is intended to stay in place for many years, but at least last for 6 to 12 months, it could have many other applications than only treating GERD or obesity.

Optionally, it is possible to proceed without spraying the niche with the biopsies with a fibrin clotting device (for example Tisseel by Baxter) before placing the stent on the niche. However, doing so, at least in certain circumstances, may be prudent to ensure that the stent stay in position.

As shown in FIGS. 20D and 20E, in one optional embodiment, a rim, flange, or belt 51′ can extend around the top of the ring of the Therapeutic GARD. The rim 51′ can extend radially outwardly at least slightly beyond the outer wall of the ring. In one optional embodiment, the ring 51′ can be about or exactly 2 mm wide. In one optional embodiment, since the magnet 50′ on the stent 42′ will be 1 mm thick as well as the vertical magnets 50′ on the ring, the rim 51′ will avoid food to pass too easily between the ring of the Therapeutic GARD and the stent where the food could stagnate and potentially cause an infection.

The following exemplary embodiments further describe optional aspects of the presently disclosed technology and are part of this Detailed Description. These exemplary embodiments are set forth in a format substantially akin to claims (each set including a numerical designation followed by a letter (e.g., “A,” “B,” etc.), although they are not technically claims of the present application. The following exemplary embodiments refer to each other in dependent relationships as “embodiments” instead of “claims.”
1A. A method for maintaining a medical device in place in a lumen of a hollow organ of a patient for a period of months or years without using a metal or bioresorbable stent.
1B. A method for placement of a medical device in a lumen of the gastrointestinal tract of a patient using a flexible endoscope having at least one 2.8 mm working channel for placement of a guidewire, biopsy forceps, endoscopic mucosal resection or endoscopic submucosal dissection devices as well as injection and spraying devices and aspiration of fluids and aspiration of air or blood or secretions.
1C. A method for placement of a permanent medical device in a wall of an esophagus of a patient using a first short-term device that is similar to a final device but without incorporation in the wall of the esophagus to evaluate safety and efficacy of the final device before permanent placement in the wall of the esophagus of the final device.
1D. A temporary device using essentially pressure on a wall of a lumen of an esophagus after calibration of a size of the esophagus that can be easily removed through the mouth after a period of one day to 1 month, usually 2 to 3 weeks.
1E. A temporary device with a thick ring leaving a niche on an esophageal wall once removed that is used to place a soft mesh ring of one of long-term Therapeutic-GARDs.
1F. A method for placement of medical devices using mucosal resection with standard biopsies or larger and deeper pieces of an esophageal wall using endoscopic mucosal resection (EMR) or endoscopic submucosal dissection (ESD) removing esophageal mucosa and having the wall of the esophagus bleed, then spraying plasma rich platelet solution obtained from the patient's blood prior to placement of the devices to help a longer-term device incorporate within to the esophageal wall.
1G. A method of isolating a portion of the esophagus to place temporary or permanent medical devices in a wall of the esophagus using biopsies to make the wall of the esophagus bleed adding platelet rich plasma to help adhesion and incorporation of medical devices in the esophageal wall made with an incorporated upper mesh ring and tubular devices placed in the luminal wall to treat at least one of Gastroesophageal reflux disease (GERD) or obesity.
1H. A method of isolating a portion of an esophagus of a patient to place temporary or permanent medical devices in a wall of an esophagus using local injection of botulinum toxin to decrease peristaltic contraction of the esophagus for a few weeks or months to keep the device in place to help a permanent device stay in position.
1I. A temporary device leaving a pressure niche on a wall of an esophagus of a patient that will be used to place longer term devices for treatment of at least one of Gastroesophageal Reflux Disease (GERD) or excess body weight.
1J. A method of using a combination of biopsies of an esophageal epithelium to obtain esophageal cells for culture and reinjection at a later endoscopy or immediately for repairing the esophageal epithelium resected to place a net or mesh ring within a wall of esophagus.
1K. A method of using culture of esophageal cells for reinjection at a later endoscopy or immediate reinjection of esophageal cells in platelet rich plasma (PRP) with calcium gluconate as a culture medium in vivo for repairing esophageal epithelium resected to contain a net or mesh ring in a wall of an esophagus of a patient that supports medical devices in a lumen of the esophagus.
1L. A method of using bleeding and injection of autologous platelet rich plasma (PRP) solution in an esophagus of a patient to help heal lesions caused by mucosal resection to obtain epithelial cells for culture or incorporation of a mesh net ring in a esophageal wall.
1M. A method of using platelet rich plasma with small fragments of about 1 mm of epithelial mucosa to culture an epithelium on a luminal side of a mesh ring so as to repair the epithelium and help incorporate safely the mesh ring at a flexible endoscopy to support luminal devices.
1N. A method of using platelet rich plasma with small fragments of epithelial mucosa to culture an epithelium on a luminal side of a mesh ring so as to repair the epithelium and help incorporate safely the mesh ring at a flexible endoscopy to support luminal devices to treat gastroesophageal reflux.
1O. A method of using platelet rich plasma with small fragments of epithelial mucosa to culture an epithelium on an external side (submucosal and muscular side of the esophagus) of a mesh ring so as to repair a wall and help incorporate safely the mesh ring using cultures of epithelial cells or culture of fibroblasts from a lamina propria obtained at flexible endoscopy to support luminal devices.
1P. A method of using platelet rich plasma with small fragments of epithelial mucosa to culture a epithelium on a luminal side of a mesh ring so as to repair the epithelium and help incorporate safely the mesh ring at flexible endoscopy to support luminal devices to treat gastroesophageal reflux blocking reflux yet allowing vomiting (lamellar device).
1Q. A method of using platelet rich plasma with small fragments of epithelial mucosa to culture an epithelium on a luminal side of a mesh ring so as to repair a epithelium and help incorporate safely the mesh ring at flexible endoscopy to support luminal devices to treat obesity.
1R. A method of culturing esophageal cells in vitro obtained from biopsies at a time of placement of the temporary device to supplement the endogenous culture of epithelial cells if needed at the time of the second definitive device for treatment of gastroesophageal reflux or obesity.
1S. A helical ring used to deploy a soft mesh ring of a definitive device, help place the mesh ring in position and exert pressure on the mesh ring so as to pressure the mesh in position on the bleeding, coagulating mix of blood, PRP and calcium gluconate.
1T. A delivery catheter using a helical spring to put pressure on an esophageal wall side of a mesh in a niche and help the mesh adhere to a mix of blood, PRP and gluconate calcium in the niche and a balloon on a delivery to press on the luminal epithelial cell layer after epithelial cells with PRP have been sprayed on the mesh to help sandwich the mesh in the wall of the esophagus.
1U. A soft mesh ring comprising or consisting of polypropylene, polyester, polyethylene terephthalate (PET), polytetrafluoroethylene (PTFE), polyvinylidene fluoride (PVDF), synthetic compounds integrating animal collagen components, and mixtures thereof, among others. 1V. The use of a delivery device to insert a definitive device and a mesh ring into a patient, the use comprising releasing the definitive device and the mesh ring in position under endoscopic control.
1W. The use of a balloon to introduce a definitive device, the use comprising inflating the mesh net ring in position and exert pressure on the mesh before removing the balloon to allow cultured endogenous or exogenous autologous epithelial cells to be injected to repair and restore original esophageal epithelium.
1X. A mesh ring that is radio-opaque to localize the mesh ring with fluoroscopy without repeating a gastroscopy.
1Y. A glue used to have the mesh of the ring adhere to the tubular part of a Gastro-intestinal Anti-Reflux Device that is radio-opaque by mixing silicon glue with a radio-opaque substance.
1Z. A glue inducing adhesion of a mesh of a ring to a tubular part of a Gastro-intestinal Anti-Reflux Device that is radio-opaque by mixing silicon glue with a radio-opaque substance such as barium sulfate.
1AA. A tubular or lamellar GARD made of a medical grade plastic.
1AB. A tubular or lamellar GARD made of a medical grade implantable silicone.
1AC. A method of allowing integration of a mesh ring using platelet rich plasma (PRP) obtained from a patient's own blood, wherein a mesh ring is integrated within a wall of the patient's esophagus between the PRP added to the biopsy sites of the wall of the esophagus that helps coagulation and grips the mesh on an external side of the mesh ring, wherein esophageal cell wall stem cells that are obtained from biopsies and are reinjected on an internal or luminal side of the mesh ring.
1AD. A method of preventing displacement of a prosthesis, optionally a stent and optionally formed of nitinol, the method comprising:
   inserting the prosthesis into a preexisting passageway of a living organism, the prosthesis contacting an interior wall of the passageway; and
   applying plasma, optionally platelet rich plasma (PRP), to at least one of the wall and the prosthesis so that the prosthesis becomes integrated into the wall of the passageway.
1AE. A system comprising:
   a prosthesis, optionally a stent and optionally formed of nitinol, configured to contact or engage an interior wall of a preexisting passageway of a living organism, and
   plasma, optionally platelet rich plasma (PRP), for applying to at least one of the wall and the prosthesis so that the prosthesis becomes integrated into the wall of the passageway.
2AE. The system of embodiment 1AE, wherein the system is configured to prevent displacement of the prosthesis in the passageway.
3AE. The system of embodiment 1AE or 2AE, wherein the plasma is sprayed or injected.
4AE. The system of embodiment 1A, 2AE, or 3AE, wherein the prosthesis is sutured to at least a portion of the passageway.
5AE. The system of embodiment 1A, 2AE, 3AE, or 4AE, wherein the plasma is compressed onto the prosthesis.
1AF. A method where biopsies are made all around a niche made by a first short-term device after the first short-term device has been removed and a stent is placed in the biopsy bleeding niche and held in place by the natural normal coagulation of the biopsies that cover the stent.
1AG. A method where biopsies are made all around a niche made by a first short-term device after the first short-term device has been removed and a stent is placed in the biopsy bleeding niche and held in place by adding biological or chemical compounds such as PRP, calcium gluconate, methylene blue and/or fibrin glue.
1AH. Methods where a stent is linked to a final long-term device by glue, knots, magnets, or simply by its own pressure conferred by the helical spring.
1AI. A method where a nitinol ring with an inner wall that is smooth and an outer wall that has holes is attached to a vertical part of a stent by one or more knots.
1AJ. A method where the natural healing process of the niche and biopsies of the esophageal wall will integrate the stent in the esophageal wall with stem cells, inflammatory and immunological cell.
1AK. A method where in addition to a stent a silicon ring is knotted to the stent with an inner smooth surface and an outer surface with holes is an intermediary device between the fixed stent and a “semi-permanent” long-term Therapeutic device in the lumen of the esophagus, that can be removed and replaced if acid, bile, or food alters the long-term device after a number of months or years in place with a simple endoscopic procedure of removing the old Therapeutic device and replacing the new Therapeutic device through the mouth.
1AL The type of Therapeutic device can be adapted to the patient's clinical condition (lamellar to tubular or vice-versa) or more or less lamellae if the patient is at more or less risk of vomiting (more lamellae are thinner and will reverse more easily in case of vomiting), fewer lamellae will be more efficient in blocking reflux in the patient needs it), the tubular Therapeutic device being the most efficient in blocking GERD but will block vomiting proportionally to the length of the tube, the longer the tube as in GARD for Obesity, the more the tube will collapse in the stomach and will not allow vomiting as is the case in gastric-bypass operations for obesity.
1AM. A method where three pressures exerted on the devices reach an equilibrium namely the pressure of the esophageal wall cells on the stent to push the stent into the lumen of the esophagus (that is the pressure from the wall of the esophagus toward the lumen of the esophagus), the counter-pressure exerted by the helical spring and the silicone ring of the device from the lumen towards the wall of the esophagus and the injection of botulinum toxin in the wall of the esophagus right above the device that blocks at least for a few weeks, as described in U.S. patent application Ser. No. 16/610,612 and granted as U.S. Pat. No. 11,571,289 as of Feb. 7, 2023, which is hereby incorporated by reference. After a few weeks, the stent should have been included in the wall of the esophagus by the inflammatory cells who have started to scar the stent in the wall of the esophagus, a natural occurrence that the body uses to isolate a foreign body that it cannot expel.
1AN. A method where magnets or parts of ferromagnetic sheets are placed in the stent facing the lumen to strengthen the contact with the vertical band magnets or ferromagnetic sheet on the external part of the Therapeutic Magnetic GARD.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the present presently disclosed technology as defined by the appended claims.

1. A stent configured to be placed in a patient to treat any of a variety of ailments, the stent comprising:
a plurality of spaced-apart horizontal supports;
a plurality of spaced-apart vertical supports, each of the plurality of spaced-apart vertical supports extending from a bottom-most one of the plurality of spaced-apart horizontal supports to top-most one of the plurality of spaced-apart horizontal supports; and
a plurality of spaced-apart connectors, each connector being positioned between a pair of the plurality of spaced-apart horizontal supports and a pair of the plurality of spaced-apart vertical supports.

2. The stent of claim 1, wherein the stent is configured to be placed in an esophagus of the patient.

3. The stent of claim 1, wherein adjacent pair of the plurality of spaced-apart horizontal supports are equidistantly spaced-apart.

4. The stent of claim 1, wherein adjacent pair of the plurality of spaced-apart vertical supports are equidistantly spaced-apart.

5. The stent of claim 1, wherein adjacent pair of the plurality of spaced-apart connectors are equidistantly spaced-apart.

6. (canceled)

7. The stent of claim 1, wherein the stent is configured to be attached to a tubular part by adhesive.

8. The stent of claim 1, wherein the stent is configured to be attached to a ring.

9. The stent of claim 7, wherein a helical spring is not employed during use of the stent in the patient.

10. The stent of claim 1, wherein the stent is configured to be attached to a ring by surgical sutures with knots.

11. The stent of claim 1, wherein the stent is configured to be attached to at least one of a ring and a tubular part by one or more magnets.

12. The stent of claim 11, wherein the one or more magnets include a plurality of spaced-apart magnets, each magnet extending over two or more of the plurality of spaced-apart horizontal supports or over two or more of the plurality of spaced-apart vertical supports.

13. The stent of claim 12, wherein each magnet is attached to the inside of the stent.

14. The stent of claim 12, wherein each magnet is attached to the outside of the stent.

15. The stent of claim 1, wherein one or more spaced-apart magnets are attached to at least one of the horizontal supports or at least one of the vertical supports.

16. The stent of claim 1, wherein the stent is reconfigurable between an expanded configuration and a folded configuration.

17. A method of minimally-invasive treatment of at least one of reflux disease and obesity of a patient, the method comprising:
inserting a stent through a mouth of the patient, the stent including a plurality of spaced-apart horizontal supports, a plurality of spaced-apart vertical supports, and a plurality of spaced-apart connectors; each of the plurality of spaced-apart vertical supports extending from a bottom-most one of the plurality of spaced-apart horizontal supports to top-most one of the plurality of spaced-apart horizontal supports; and each connector being positioned between a pair of the plurality of spaced-apart horizontal supports and a pair of the plurality of spaced-apart vertical supports.

18. The method of claim 17, further comprising:
attaching one or more spaced-apart magnets to the stent.

19. The method of claim 17, further comprising:
attaching the stent to a tube with adhesive prior to inserting the stent into the patient.

20. The method of claim 17, further comprising:
attaching the stent to a ring using surgical thread to create a plurality of knots around the stent and the ring prior to inserting the sent into the patient.

21. (canceled)

22. A method of stopping reflux or obesity of a patient, the method comprising:
using the pressure-counter pressure principle, where the pressure on a stent from a body to push the stent in a lumen of an esophagus of a patient is countered by pressure of a ring of a device with a helical spring for horizontal pressures, and
injecting botulinum toxin above the stent in a wall of the esophagus after placement of the stent,
wherein a therapeutic device counteracts peristalsis contraction of the esophagus to push the therapeutic device down into a stomach of the patient,
wherein balance is kept long enough for inflammatory cells to create a scar around the stent and the helical ring.
